(19) United States
(12) Patent Application Publication
(10) Pub. No.: US 2026/0014262 A1
(43) Pub. Date: Jan. 15, 2026

(54) ANTIMICROBIAL BLOCK POLYPEPTIDE, THERAPEUTIC COMPOSITION INCLUDING THEREOF, AND METHOD FOR TREATING BACTERIAL GASTROINTESTINAL INFECTION USING THEREOF

(71) Applicant: National Cheng Kung University, Tainan (TW)

(72) Inventors: Jeng-Shiung JAN, Tainan (TW); Yuan-Pin Hung, Tainan (TW); I-Fan Lin, Kaohsiung (TW)

(21) Appl. No.: 19/265,342

(22) Filed: Jul. 10, 2025

Related U.S. Application Data
(60) Provisional application No. 63/669,248, filed on Jul. 10, 2024.

(51) Int. Cl. A61K 47/60; A61K 9/00; A61K 38/04; A61K 47/61; A61P 1/00; A61P 31/04

(52) U.S. Cl. CPC A61K 47/60; A61K 9/0031; A61K 9/0053; A61K 38/04; A61K 47/61; A61P 1/00; A61P 31/04

(57) ABSTRACT

The present invention discloses an antimicrobial block polypeptide, a therapeutic composition comprising the antimicrobial block polypeptide, and a method for treating bacterial gastrointestinal infection by administering the therapeutic composition. Antimicrobial block polypeptide comprises a first positively charged peptide segment demonstrating potent sporicidal effects on gastrointestinal infectious bacteria. The therapeutic composition may further contain an enzyme-targeting macromolecule, where the antimicrobial block polypeptide docks on negatively charged functional group thereof. Such enzyme-targeting macromolecule-based therapeutic agent delivery system enhances sustainability of the antimicrobial block polypeptide in the body of a subject when administered orally. Moreover, the antimicrobial block polypeptide is capable of suppressing bacterial gastrointestinal infection without disrupting intestinal microbiota.

### CROSS-REFERENCE TO RELATED APPLICATION

This application claims the benefit of U.S. Patent application Ser. No. 63/669,248, filed Jul. 10, 2024. The disclosure of the above application is incorporated herein in its entirety by reference.

### FIELD OF THE INVENTION

The present invention relates to a block polypeptide, particularly relates to a block polypeptide presenting antimicrobial activity. The present invention also relates to a gel-form delivery system to deliver the block polypeptide, and such gel-form delivery system incorporates at least the block polypeptide and an enzyme-targeted macromolecule capable of protecting and directing the block polypeptide to cites in need. The present invention further relates to a method for treating bacterial gastrointestinal infection by suppressing pathogenic bacterial growth and modulating intestinal microbiota.

### BACKGROUND OF THE INVENTION

The escalating threat of antibiotic-resistant pathogens demands innovative antimicrobial approaches in chemical engineering. Among these, Clostridioides difficile, an anaerobic gram-positive bacillus, presents a particularly challenging healthcare pathogen due to its unique spore-forming capabilities and toxin production. The spores of C. difficile exhibit exceptional resistance to conventional antibiotics and disinfectants, facilitating persistent and recurrent C. difficile infection (CDI). The major virulent factors of C. difficile are toxins A and B (encoded by the tcdA and tcdB genes) and the binary C. difficile transferase toxin (CDT). These toxins disrupt colonic epithelial integrity, enhance bacterial adherence, and trigger inflammation and colitis.

As a leading cause of healthcare-associated infections, CDI management remains complex. The infection initiates with the germination of C. difficile spores into vegetative cells that colonize the host colon. Additionally, C. difficile biofilm formation enhances colony persistence, antimicrobial resistance, and virulence. Current treatments, including vancomycin and fidaxomicin, cannot effectively eliminate spores or biofilms, and vancomycin use often disrupts the gut microbiome, increasing recurrence risk.

### SUMMARY OF THE INVENTION

Limitations as addressed hereinabove underscore the need for novel antimicrobials capable of targeting a variety of forms of bacterial gastrointestinal infection, such as C. difficile, while preserving the intestinal microbiota.

Recent advances in polymer engineering have highlighted block polypeptides, including linear, branched or star-shaped block polypeptides, based on positively charged peptide segments as promising antimicrobial candidates. These polypeptides, synthesized via ring-opening polymerization (ROP) with biocompatible, hydrophilic, and noncytotoxic polyglycerol dendrimers (PGDs) as core initiators. Their unique architecture, combining cationic and hydrophobic residues, enables potent broad-spectrum antimicrobial activity.

In the first aspect, the present invention provides an antimicrobial block polypeptide comprising a first positively charged peptide segment consisting of 5 to 20 substituted or unsubstituted amino acids selected from the group consisting of L-lysine, L-arginine, L-histidine L-omithine, and L-homoarginine.

Adapted in various embodiments, the antimicrobial block polypeptide is a linear block polypeptide, a branched block polypeptide or a star-shaped block polypeptide.

In some embodiments, the antimicrobial block polypeptide, being a branched block polypeptide or a star-shaped block polypeptide, comprises a second positively charged peptide segment consisting of 5 to 20 substituted or unsubstituted amino acids selected from the group consisting of L-lysine, L-arginine, L-histidine L-omithine, and L-homoarginine.

In some preferred embodiments, the star-shaped block polypeptide comprises; a polyol initiator or a dendrimer initiator as a core; and at least 3 arms radiating from the core, wherein the first positively charged peptide segment links to at least one arm of the 3 arms, and the second positively charged peptide segment links to at least one another arm of the 3 arms.

Preferably, the first positively charged peptide segment is consisting of substituted or unsubstituted L-lysine or L-homoarginine; the second positively charged peptide segment is consisting of substituted or unsubstituted L-lysine or L-homoarginine.

More preferably, the star-shaped block polypeptide has 3 to 24 arms.

In one another aspect, the present invention provides a therapeutic composition, comprising the aforementioned antimicrobial block polypeptide.

Preferably, the therapeutic composition is an enterally absorbable composition.

In some preferred embodiments, the therapeutic composition further comprises an enzyme-targeting macromolecule, comprising: a backbone; and a negatively charged functional group residing on the backbone, wherein the antimicrobial block polypeptide docks on the enzyme-targeting macromolecule via electrostatic interaction between the first positively charged peptide segment and the negatively charged functional group.

Preferably, the backbone comprises glycosaminoglycan selected from a group consisting of Hyaluronic Acid (HA), Chondroitin Sulfate (CS), Dermatan Sulfate (DS), Keratan sulfate (KS), Heparan sulfate, (HS), and Heparin (HP).

Preferably, the negatively charged functional group is selected from a group consisting of thiol, thiolate, thioacid, thiophosphate ester, and selenol.

More preferably, the therapeutic composition is an oral composition.

In further one another aspect, the present invention provides a method for treating bacterial gastrointestinal infection, comprising: administering an effective amount of the aforementioned therapeutic composition to a subject in need thereof, wherein the bacterial gastrointestinal infection comprises pseudomembrane colitis, gastroenteritis, diarrhea, toxic megacolon or gastrointestinal perforation.

Preferably, the therapeutic composition reduces population, spore number or biofilm of a pathogen inducing the bacterial gastrointestinal infection.

### BRIEF DESCRIPTION OF THE DRAWINGS

FIGS. 1A to 1D are schematic illustration of star-shaped polypeptides.

FIGS. 1E to 1F are schematic illustration of the microgels formation.

FIGS. 1G to 1H present synthesis scheme of G2-PLL and G3-PLL using polyglycerol dendrimers (PGD) of generation 2 and 3 as core initiators, respectively, where ZLL NCA: Z-L-Lysine N-carboxyanhydride; ROP: ring-opening polymerization; TMG: 1,1,3,3-tetra-methylguanidine; HBr, hydrogen bromide; NMR: nuclear magnetic resonance; GPC-LS: gel permeation chromatography-light scattering.

FIG. 1I illustrates 2D ¹H-¹³C HSQC NMR analysis of (left) PGD-G3 initiator in D₂O and (right) 24s-PZLL₁₀ in TFA-d₁.

FIG. 1J illustrates synthesis scheme of cysteamine-modified hyaluronic acid.

FIGS. 1K to 1O illustrate characterization of HA/G3-PLL₉ microgels, wherein FIG. 1K demonstrates the particle size distribution of HA/G3-PLL₉ microgels, FIG. 1L demonstrates the percentage of size changes for HA/G3-PLL₉ microgels under hyaluronidase treatment (0.2 mg mL⁻¹); FIGS. 1M to 1O demonstrate the percentage of size changes for HA/G3-PLL₉ microgels under various pH conditions.

FIG. 2A is a schematic illustration of G3-PLL₉ structure showing a dendrimer initiator (orange), carbon (black), oxygen (red), and branching poly-L-lysine (PLL, blue; detailed chemical structure shown in the inset).

FIG. 2B demonstrates time-kill curve of C. difficile treated with G3-PLL₉ at varying concentrations versus vancomycin at its minimum inhibitory concentration (1 μM). (n=2, repeated thrice)

FIG. 2C demonstrates dose-dependent disruption of C. difficile biofilm by G3-PLL₉, OD₅₇₀ values indicate biofilm mass; vancomycin (1 μM) serves as a control. (n=3, repeated twice)

FIG. 2D demonstrates viable C. difficile counts after G3-PLL₉ treatment of spores followed by germination with taurocholic acid. (n=2, repeated thrice)

FIG. 2E demonstrates fluorescence microscopy of C. difficile spores treated with G3-PLL₉. Green: viable spores (SYTO-9), red: non-viable spores (PI), blue: G3-PLL₉ (Alexa Fluor® 350). White spots indicate non-viable spores interacting with G3-PLL₉. Scale bars: 20 μm.

FIG. 2F demonstrates quantification of fluorescence microscopy images showing the percentage of viable and non-viable spores relative to the total spore count. (n=10 per test condition)

FIG. 2G are scanning electron microscopy images of C. difficile cells and spores, untreated (left) and treated with G3-PLL₉ for different durations. Early roughening of spore outer layer (10 mins) and size reduction (30-60 mins) are observed.

FIG. 3A is the experimental design of In vivo therapeutic efficacy of G3-PLL₉ in a mouse model of C. difficile infection (CDI).

FIG. 3B demonstrates percentage of weight change relative to the day of infection.

FIG. 3C are images of representative gross morphology of mouse cecum (Ce) and colon (Co) in each group.

FIGS. 3D to E are bar charts representing therapeutic response indicators: colon length and cecum weight, respectively.

FIGS. 3F to H are histopathological analyses of colon sections from CDI-infected mice: No treatment (FIG. 3F), vancomycin-treated (FIG. 3G), and G3-PLL₉-treated (FIG. 3H) groups. (scale bars: 10 μm)

FIG. 3I is a bar chart of histology injury scores based on tissue damage, mucosal edema, and neutrophil infiltration; six high-power field images were scored per sample.

FIGS. 3J to 3M are bar charts representing safety assessment of G3-PLL9 through biochemistry tests: blood urea nitrogen (BUN) (FIG. 3J), creatinine (FIG. 3K), aspartate aminotransferase (AST) (FIG. 3L), and alanine transaminase (ALT) (FIG. 3M).

FIGS. 3N and 3O are bar charts representing therapeutic response indicators upon oral and enema delivery of G3-PLL9: colon length and cecum weight, respectively.

FIG. 4A presents a schematic protocol of the recurrent CDI mouse model.

FIGS. 4B to 4C are line charts presenting percentage changes in body weight (B) and survival curve (C) over 15 days relative to the day of infection, respectively. Shaded areas indicate treatment periods, and the arrow marks symptom recurrence.

FIG. 4D is a bar chart presenting stool tcdB levels measured by real-time PCR to detect the presence of C. difficile toxins.

FIGS. 4E to 4F are bar charts presenting therapeutic response indicators: cecum length and cecum weight, respectively.

FIG. 5A presents synthesis of HA/G3-PLL₉ microgels via electrostatic interaction. Cys, cysteamine.

FIG. 5B is a bar chart presenting hyaluronidase levels in the colon of uninfected and CDI-infected mice.

FIG. 5C presents experiment design of oral delivery of cysteamine-modified hyaluronic acid/G3-PLL₉ microgel.

FIGS. 5D to 5E are bar charts presenting therapeutic response indicators: colon length and cecum weight, respectively.

FIG. 5F is a bar chart presenting percentage change in body weight compared to the day of infection.

FIGS. 5G to 5I presents histopathological analyses of colon sections from CDI-infected mice: No treatment (FIG. 5G), vancomycin-treated (FIG. 5H), and HA/G3-PLL₉-treated (FIG. 5I) groups. (scale bars: 100 μm).

FIG. 5J is a bar chart presenting histology injury scores based on tissue damage, mucosal edema, and neutrophil infiltration, with six high-power field images per sample.

FIGS. 5K to 5M are bar charts presenting safety assessment of HA/G3-PLL₉ via blood urea nitrogen (BUN) (FIG. 5K), creatinine (FIG. 5L), and alanine transaminase (ALT) (FIG. 5M) levels.

FIG. 6A are stacked bar charts showing the relative abundance of bacterial genera across No infection, No treatment, vancomycin, G3-PLL₉ 1 μM (enema), and hyaluronic acid (HA)/G3-PLL, microgels 2.5 μM (oral) groups.

FIG. 6B is a box plot of alpha diversity (Shannon entropy) for each group. Statistical analysis was conducted using the Kruskal-Wallis test. *p<0.05, **p<0.01.

FIG. 6C presents principal coordinates analysis (PCoA) based on Bray-Curtis dissimilarity, illustrating beta diversity. Each point represents the microbial community of an individual sample, with proximity indicating greater similarity.

FIG. 6D presents linear discriminant analysis Effect Size (LEfSe) highlighting bacterial species with significantly different abundances among groups. LEfSe uses a non-parametric Kruskal-Wallis test for significance and linear discriminant analysis to estimate effect sizes.

### DETAILED DESCRIPTION OF THE INVENTION

The following embodiments are provided to further illustrate the present invention in detail. Upon reviewing the contents of this specification, a person of ordinary skill in the art will readily appreciate the advantages and technical effects of the present invention, and will be able to implement the disclosure or apply it in various alternative embodiments. Accordingly, modifications, substitutions, or alterations to the embodiments described herein may be made without departing from the spirit and scope of the present invention. Moreover, any element or method disclosed in the present invention may be combined with any other element or method described in any of the embodiments without limitation, unless expressly stated otherwise.

As used herein, the singular forms “a,” “an,” and “the” include plural referents unless expressly and unequivocally limited to one referent. The term “or” is used interchangeably with the term “and/or” unless the context clearly indicates otherwise.

The terms “peptide(s),” “polypeptide(s)” and “block polypeptide(s)” are used herein to designate a series of amino acid residues that have multiple amino acid residues, such as 10 to 40 amino acids in length, 10 to 30 amino acids in length or 10 to 25 amino acids in length, for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids in length. “Block polypeptide(s)” refers to a peptidyl structure comprising an amino acid segment consisting of: a repeat of one type of amino acid; or a repeat of amino acids with a single electrical charge.

Peptides, polypeptides, or block polypeptides (hereinafter referred to as peptides) in the present application may include the standard 20 α-amino acids that are used in protein synthesis by cells (i.e. natural amino acids), as well as non-natural amino acids (may be found in nature, but not used in protein synthesis by cells, e.g., orithine, citrulline, and sarcosine, or may be chemically synthesized), amino acid analogs, and peptidomimetics. The amino acids may be D- or L-optical isomers. The peptides may be formed by a condensation or coupling reaction between the α-carbon carboxyl group of one amino acid and the amino group of another amino acid. The terminal amino acid at one end of the chain (amino terminal) therefore has a free amino group, while the terminal amino acid at the other end of the chain (carboxy terminal) has a free carboxyl group. Alternatively, the peptides may be non-linear, branched peptides or cyclic peptides. Moreover, the peptides may optionally be modified or protected with a variety of functional groups or protecting groups, including on the amino and/or carboxy terminus.

The amino acid residues in the peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

The peptides are antimicrobial themselves and serve as antibiotics or antimicrobial agents, or serve as active compounds in the antimicrobial composition.

The term “antimicrobial” refers to suppressing microbe population growth upon administration of the antimicrobial block polypeptide, in comparison to a control group without administration thereof. In some embodiments, the antimicrobial block polypeptide suppresses microbe population growth by disrupting microbial cell walls, suppressing microbial cell wall synthesis, inhibiting microbe reproduction or reducing number of microbial spores, but not limited to this. Antimicrobial effects can be evaluated by well-acknowledged antimicrobial assays, as demonstrated in the following embodiments in the present invention.

The “bacterial gastrointestinal infection” refers to observable microbial population in gastrointestinal tract of a certain subject. With growth of the microbial population, malignant response may be activated in gastrointestinal tract, leading to disease or syndrome exemplified by pseudomembrane colitis, gastroenteritis, diarrhea, toxic megacolon or gastrointestinal perforation.

The microbe involved in the present invention includes at least a pathogen causing the bacterial gastrointestinal infection. The pathogen refers to Salmonella, Staphylococcus, Escherichia, Vibrio, or Clostridium. The antimicrobial block polypeptide presents particularly significant antimicrobial effects on Clostridium leading to bacterial gastrointestinal infections. Examples of the bacteria belonging to the genus Clostridium include, but are not particularly limited to, Clostridium botulinum, Clostridium difficile, Clostridium perfringens, Clostridium aldenense, Clostridium asparagiforme, Clostridium bolteae, Clostridium citroniae, Clostridium clostridioforme, Clostridium lavalense, Clostridium nexile, Clostridium populeti, and Clostridium symbiosum.

The peptides may contain or may be modified to contain, functional groups to which a water-soluble polymer may be attached, either directly or through a spacer moiety or linker. Functional groups include, but are not limited to, the N-terminus of the peptide, the C-terminus of the peptide, and any functional groups on the side chain of an amino acid, e.g. lysine, cysteine, histidine, aspartic acid, glutamic acid, tyrosine, arginine, serine, methionine, and threonine, present in the peptides.

Furthermore, modifications can occur anywhere in the peptides, including the peptide backbone, the amino acid side-chains, and the N- or C-termini.

“Optional” or “optionally” means that the subsequently described circumstance may or may not occur, so that the description includes instances where the circumstance occurs and instances where it does not.

The term “about” as used herein when referring to the numerical value is meant to encompass variations of ±20%, ±10%, ±5%, ±1%, ±0.5%, or ±0.1% from the numerical value. Such variations in the numerical value may occur by, e.g., the experimental error, the typical error in measuring or handling procedure for making compounds, compositions, concentrates, or formulations, the differences in the source, manufacture, or purity of starting materials or ingredients used in the present invention, or like considerations.

As used herein, the terms “comprise,” “comprising,” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

The therapeutic composition of the present invention may be administered intravenously, intra-arterially, intra-peritoneally, intramuscularly, intradermally, intratumorally, orally, dermally, nasally, buccally, rectally, enterally, vaginally, by inhalation, or by topical administration.

The therapeutic composition of the present invention may be administered to a subject including a plant or an animal, etc. The terms “subject,” “individual,” or “patient” are used interchangeably herein. The animal may be a fish, a bird, a mammal, etc., but it is not limited thereto. Examples of the mammal may include, but is not limited to, a cat, a dog, a bovine, a horse, a pig, a human, etc. In one embodiment, the therapeutic composition of the present invention may be administered to a human.

An “effective amount” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

In the first aspect, the present invention provides an antimicrobial block polypeptide (1) comprising a first positively charged peptide segment consisting of 5 to 20 substituted or unsubstituted amino acids selected from the group consisting of L-lysine, L-arginine, L-histidine L-omithine, and L-homoarginine.

The antimicrobial block polypeptide (1) may be a linear block polypeptide, a branched block polypeptide, or a star-shaped block polypeptide.

In some embodiments, the first positively charged peptide segment is consisting of substituted or unsubstituted L-lysine or L-homoarginine; preferably, the first positively charged peptide segment is selected from the group consisting of L-lysine₆, L-lysine₉, L-lysine₁₀, L-lysine₁₂, L-lysine₁₅, L-homoarginine₆, L-homoarginine₉, L-homoarginine₁₀, L-homoarginine₁₂, and L-homoarginine₁₅; more preferably, the first positively charged peptide segment is L-lysine₉ or L-lysine₁₀.

In other embodiments, the branched block polypeptide or the star-shaped block polypeptide may contain a second positively charged peptide segment consisting of 5 to 20 substituted or unsubstituted amino acids selected from the group consisting of L-lysine, L-arginine, L-histidine L-omithine, and L-homoarginine. Conceivably, the second positively charged peptide segment may be the same as or different from the first positively peptide segment in length or types of amino acids. For example, when the first positively charged peptide segment consists of 5 to 20 substituted or unsubstituted L-lysine, the second positively charged peptide segment consists of 5 to 20 substituted or unsubstituted amino acids such as L-arginine, L-histidine L-omithine, or L-homoarginine.

In preferred embodiments, the second positively charged peptide segment is consisting of substituted or unsubstituted L-lysine or L-homoarginine. Preferably, the second positively charged peptide segment is selected from the group consisting of L-lysine₆, L-lysine₉, L-lysine₁₀, L-lysine₁₂, L-lysine₁₅, L-homoarginine₆, L-homoarginine₉, L-homoarginine₁₀, L-homoarginine₁₂, and L-homoarginine₁₅.

In some embodiments, the branched block polypeptide may contain the second positively peptide segment as a branch segment linking to any one of amino acid residues except for the C-terminal amino acid or N-terminal amino acid of the first positively peptide segment.

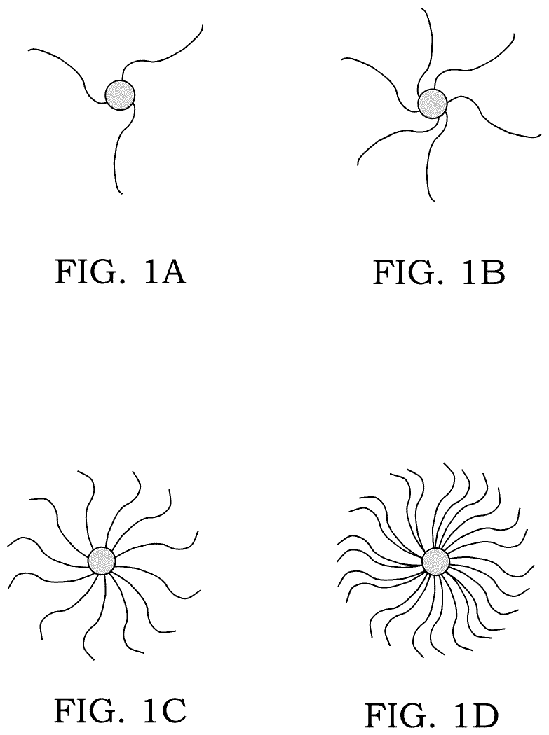

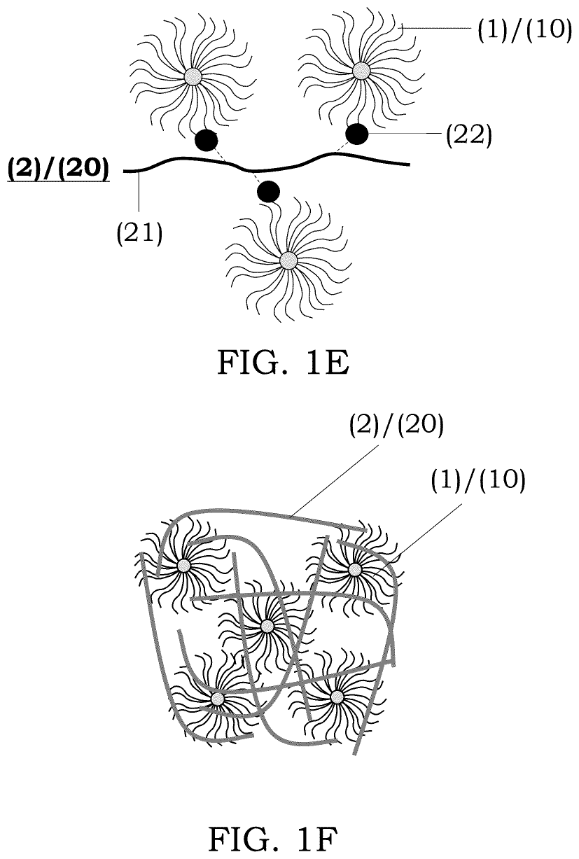

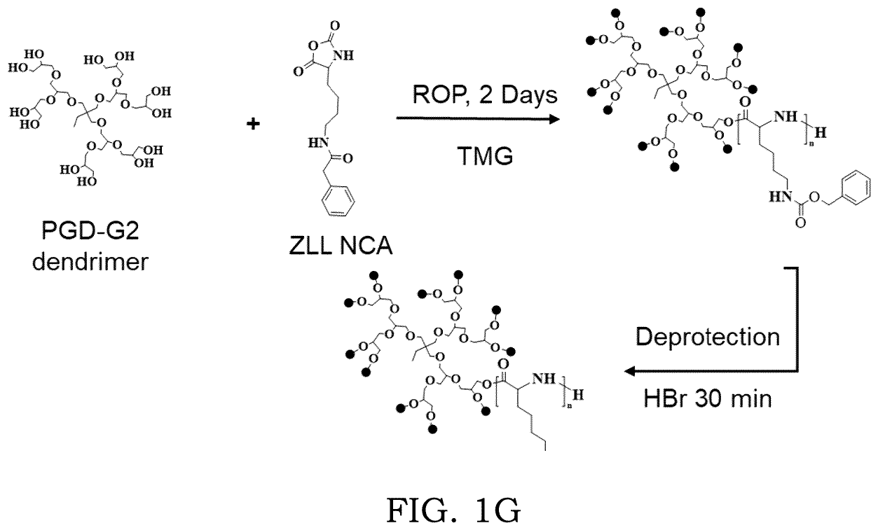

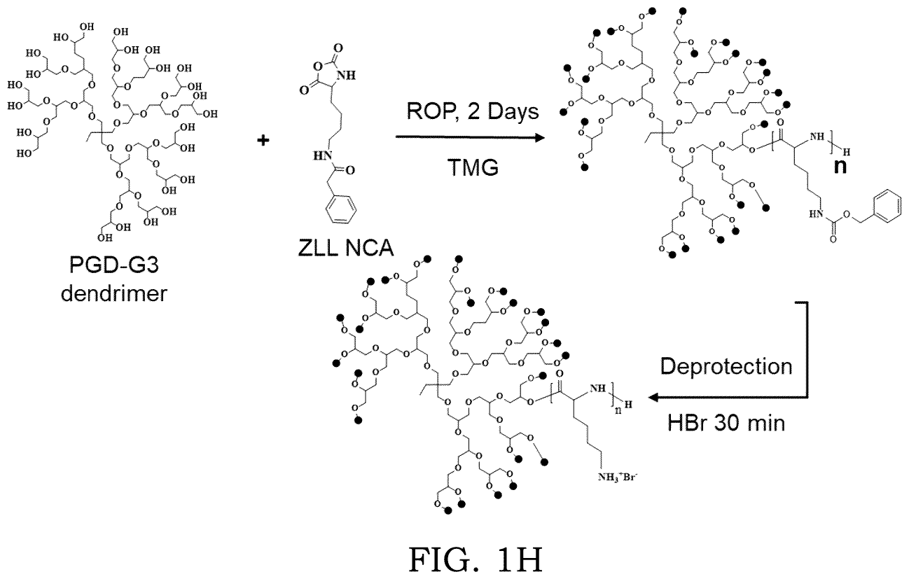

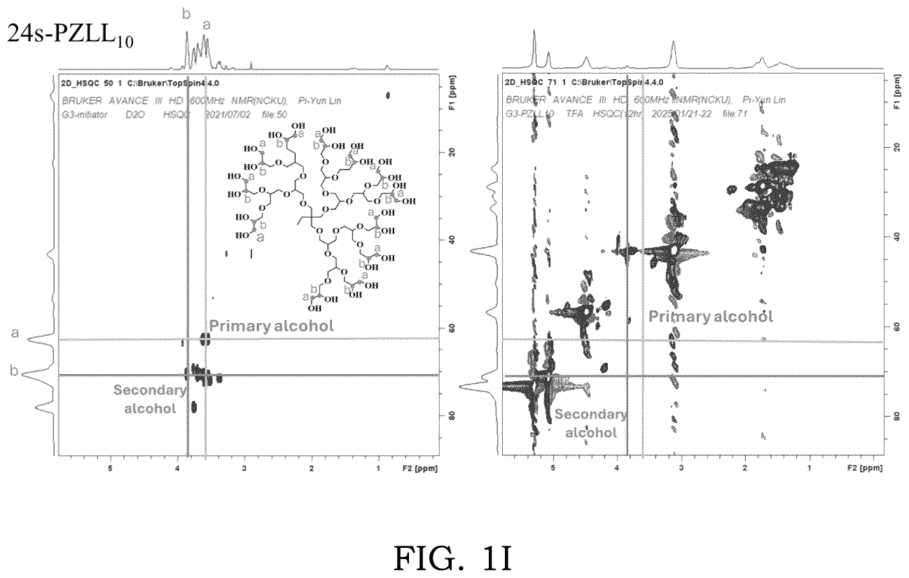

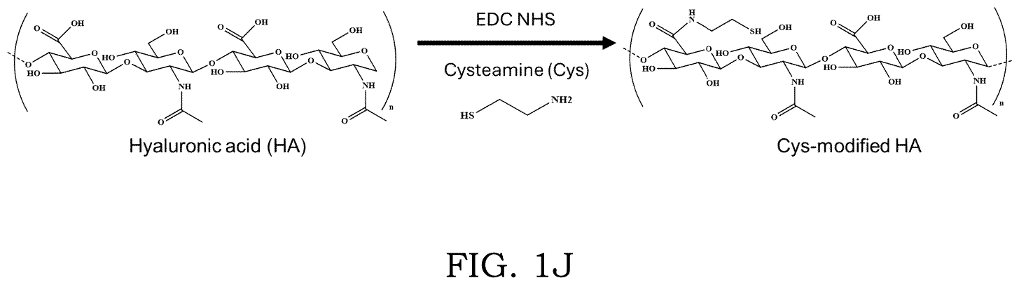

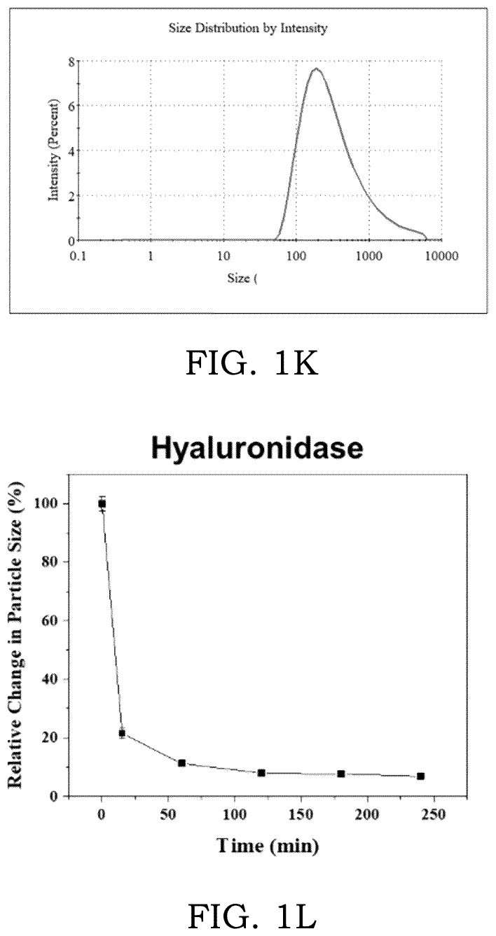

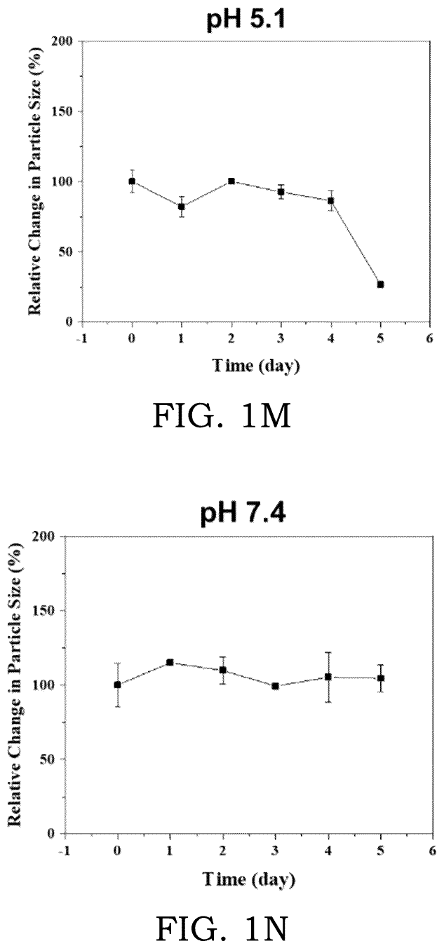

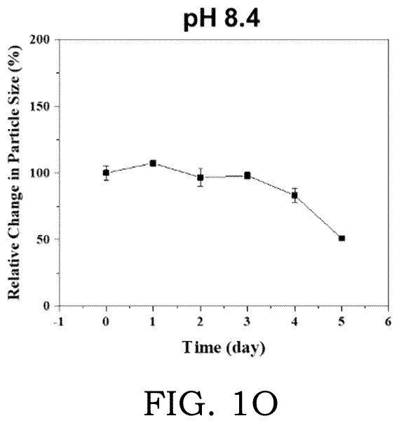

In some other embodiments, as shown in FIGS. 1A to 1D, the star-shaped block polypeptide comprises a polyol initiator or a dendrimer initiator as a core; and at least 3 arms radiating from the core. Preferably, the star-shaped block polypeptide comprises at least 3 to 24 arms, such as 3, 6, 12, or 24 arms.

In various embodiments, the first positively charged peptide segment links to at least one arm of the 3 arms, or every arm of the 3 arms. Preferably, the first positively charged peptide segment links to at least one arm of the 3, 6, 12 or 24 arms, or every arm of the 3, 6, 12, or 24 arms.

In a few embodiments, the star-shaped block polypeptide may contain the second positively charged peptide segment linking to at least one another arm of the 3 arms. Preferably, the second positively charged peptide segment links to at least one another arm of the 3, 6, 12 or 24 arms. There is no particular arrangement of the first positively charged peptide segment and the second positively charged peptide segment around the core.

In various embodiments, the polyol initiator may be 1,1,1-tris(hydroxymethyl)propane or dipentaerythritol; the dendrimer initiator may be a poly(amidoamine) (PAMAM) dendrimer, a polylysine dendrimer, a poly (propylene imine (PPI) dendrimer, a polyester dendrimer, a polyglutamic acid dendrimer, a polyaspartic acid dendrimer, a polyglycerol dendrimer, and a polymelamine dendrimer.

The block polypeptides of the present invention may be synthesized by the ring-opening polymerization (ROP) method. For example, amino acids suitable for synthesizing the segment having desired property (i.e., positively charged or hydrophobic) are selected and reacted to form N-carboxyanhydrides (NCAs). Optionally, some amino acids require an additional side chain protection step (for example, using benzyloxycarbonyl group (Z group)) and such step is performed prior to the NCA formation step. ROP is then carried out by amine initiators and NCAs to form the desired block polypeptides. If a side chain protection step has been performed previously, an additional side chain deprotection step is required after block polypeptides formation.

In some embodiment, star-shaped block polypeptide positively charged amino acids and core initiators may be synthesized by ROP, and L-lysine, L-arginine, L-histidine L-omithine, or L-homoarginine is selected to react with a core initiator including a polyol or a dendrimer to form the star-shaped block polypeptide. In certain examples, L-lysine (LL, suitable for synthesizing a positively charged segment) and polyglycerol dendrimer (PGD), suitable for serving as a radial-shaped core) are selected. L-lysine is reacted with NCA to form Z-L-Lys-NCAs (namely, L-lysine protected by a NCA group). Then, Z-L-Lys-NCAs and polyglycerol dendrimer are reacted to form PGD-P (LL-NCA)ₙ by ROP using 1,1,3,3-tetra-methylguanidine as the initiator. The star-shaped block polypeptide PGD-PLL, was then obtained by deprotection of the Z group. The n may be from 5 to 20, such as 5, 6, 7, 8, 9, 10, 11, 12, 13, 14, 15, 16, 17, 18, 19, or 20.

In the second aspect, the present invention provides a therapeutic composition comprising the preceding antimicrobial block polypeptide (1).

Optionally, the therapeutic composition may be an enterally absorbable composition or an orally intake composition.

In preferred embodiments, as shown in FIG. 1E to 1F, the therapeutic composition further comprising an enzyme-targeting macromolecule (2) serving as a vector to deliver the antimicrobial block polypeptide (1). The enzyme-targeting macromolecule (2) is preferably a saccharide-based macromolecule, and with its facilitation, targeted delivery of the therapeutic composition can be conducted when activity or amount of enzyme specific to the enzyme-targeting macromolecule (2) is increased in a bacterial infection site.

Presumably, with enzymatic digestion of the enzyme-targeting macromolecule (2), the antimicrobial block polypeptide (1) can be released.

Referring to FIG. 1E, the enzyme-targeting macromolecule (2) comprises a backbone (21) and a negatively charged functional group (22) residing on the backbone (21), wherein the antimicrobial block polypeptide (1) docks on the enzyme-targeting macromolecule (1) via electrostatic interaction between the first positively charged peptide segment and the negatively charged functional group (22).

As being a saccharide-based macromolecule, the backbone (22) may be formed of multiple saccharide bases, and each of the saccharide bases may be glucose, lactose, galactose or idose. Preferably, the backbone (21) is modified with minor negative charged functional group such as acetyl group, carboxyl group, or acetylamino group. In one embodiment, the backbone is one type of glycosaminoglycan or its salt. The glycosaminoglycan may be Hyaluronic Acid (HA), Chondroitin Sulfate (CS), Dermatan Sulfate (DS), Keratan sulfate (KS), Heparan sulfate (HS), or Heparin (HP). Preferably, the glycosaminoglycan is Hyaluronic Acid (HA).

In various embodiments, with glycosaminoglycan serving as the vector, as shown in FIG. 1F, the antimicrobial block polypeptide (1) is entrapped by one or multiple molecules of glycosaminoglycan, thereby forming a gel-like complex. Preferably, the gel-like complex is a microgel with particle size ranging from 300 to 400 nm. More preferably, the particle size of the microgel ranges from 310 to 390 nm, 320 to 380 nm, 330 to 370 nm, 340 to 365 nm, or 350 to 360 nm.

The negatively charged functional group (22), being distinct from the minor negatively charged functional group, may be thiol, thiolate, thioacid, thiophosphate ester, and selenol. Specifically, the negatively charged functional group (22) may substitute any one side group of one of the saccharide bases, or the negatively charged functional group (22) may be a functional group of a negatively charged molecule attaching and/or linked to the backbone (21). For example, the negatively charged molecule may be cysteine, methanethiolate, selenocysteine, thioacetic acid or thio-ATP. Preferably, taking the total number of the multiple saccharide bases to be the basal number, a degree of substitution (DoS) of the negatively charged functional group (22) on the backbone (21) may be at least 30% to 99%. More preferably, the degree of substitution of the negatively charged functional group (22) may be 30% to 50%, 35% to 45%, or 38% to 42%.

In certain embodiments, the enzyme-targeting macromolecule (2) may be synthesized by grafting a saccharide-based macromolecule via a carboxyl activation method. For example, when modifying a glycosaminoglycan, 1-ethyl-3-(3-dimethylaminopropyl) carbodiimide (EDC) and N-hydroxysuccinimide (NHS) are selected for carboxyl activation before the negatively charged molecule attaches or links to the activated carboxyl group. In one example, EDC, NHS, HA and cysteamine of proper molar ratio are mixed and reacted to form a Cysteamine-grafted HA (Cys-HA).

In various embodiments, apart from directing the antimicrobial block polypeptide (1) to the target site enriched of enzyme specific to the enzyme-targeting macromolecule (2), the enzyme-targeting macromolecule (2) also protects the antimicrobial block polypeptide from being degraded in the body. Moreover, the enzyme-targeting macromolecule (2) may also enable the therapeutic composition to sustainably release the antimicrobial block polypeptide (1). For example, as the therapeutic composition is administrated in a gel-like form, one skilled in the art may adjust the ratio of the enzyme-targeting macromolecule (2) to the antimicrobial block polypeptide (1) so that the release rate of the antimicrobial block polypeptide (1) can be controlled with reference to its inherent pharmacokinetic characteristics.

In a third aspect, the present invention provides a use of the antimicrobial block polypeptide for manufacturing a therapeutic composition, wherein the therapeutic composition comprises: an antimicrobial block polypeptide (10); or an antimicrobial block polypeptide (10) and an enzyme-targeting macromolecule (20). As chemical structures and properties of the antimicrobial block polypeptide (10) and the enzyme-targeting macromolecule (20) are overall the same as the antimicrobial block polypeptide (1) and the enzyme-targeting macromolecule (2) provided in the first and second aspects of the present invention, details thereof are omitted hereinafter.

In a fourth aspect, the present invention provides a method for treating bacterial gastrointestinal infection, comprising: administering an effective amount of a therapeutic composition to a subject in need thereof, wherein the therapeutic composition comprises the therapeutic composition as provided in the second aspect of the present invention.

To ensure consistency between the administration and the dose, an enterally-administrated dosage form or an orally-administrated dosage form of the therapeutic composition is preferred. The “dosage form” used herein refers to a form separated physically, being suitable to deliver a unit dose to the subject in need of therapy. Each unit dose contains a quantified amount of the antimicrobial block polypeptide (1) expected to produce therapeutic effects, and includes a drug vector if necessary. The unit dose in the present invention is determined and depends on characteristics of the antimicrobial block polypeptide (1) and specific therapeutic effects as anticipated.

In some embodiments, the therapeutic composition is administered enterally to the subject. Of note, the enterally-administrated therapeutic composition comprises the antimicrobial block polypeptide (1) with or without the enzyme-targeted macromolecule (2).

In other embodiments, the therapeutic composition is administered orally to the subject. It should be noted that the orally-administrated therapeutic composition comprises the antimicrobial block polypeptide (1) and the enzyme-targeted macromolecule (2).

The effective amount depends on the subject in need and the dosage form of the therapeutic composition. In some embodiments, the therapeutic composition is administrated enterally to the subject in need; for example, the subject in need is a mouse, and the effective amount is from 7.5×10⁻¹⁰ mol/kg to 60×10⁻¹⁰ mol/kg bodyweight of the subject; as the subject is a human, the effective amount is from 0.5×10⁻¹⁰ mol/kg to 5.0×10⁻¹⁰ mol/kg bodyweight of the subject. Taking a human adult of 60 kg, one does of the therapeutic composition is 12×10⁻¹⁰ mol to 300×10⁻¹⁰ mol. In other embodiments, the therapeutic composition is administrated orally to the subject in need; as the subject in need is a mouse, the effective amount is from 25×10⁻¹⁰ mol/kg to 200×10⁻¹⁰ mol/kg bodyweight of the subject; as the subject is a human, the effective amount is from 2.0×10⁻¹⁰ mol/kg to 16.5×10⁻¹⁰ mol/kg bodyweight of the subject. Taking a human adult of 60 kg, one does of the therapeutic composition is 120×10⁻¹⁰ mol to 990×10⁻¹⁰ mol.

In various embodiments, to ensure persistence of the antimicrobial effect, the therapeutic composition is administered to the subject in need for at least two times, with a minimum interval of 16 to 24 hours between administrations.

In various embodiments, the therapeutic composition reduces population, spore number or biofilm of a pathogen inducing the bacterial gastrointestinal infection.

In preferred embodiments, the pathogen is Clostridium difficile. In certain embodiments, the Clostridium difficile infects cecum or colon of the subject, resulting in malignant enlargement of cecum or lengthening of colon.

Exemplary embodiments of the present invention are further described in the following examples, which should not be construed to limit the scope of the present invention.

### Embodiment 1: Star-Shaped Block Polypeptides

### 1.1 Synthesis

The 12-armed and 24-armed star-shaped polypeptides, G2-PLL and G3-PLL, were synthesized via ROP using PGD of generation 2 and 3 (PGD-G2 with Mₙ=800 g mol⁻¹ and PGD-G3 with Mₙ=1689.8 g mol⁻¹) as the core initiators along with Z-L-Lysine (ZLL) N-carboxyanhydride (NCA) monomer, followed by deprotection (FIGS. 1G to 1H). 1,1,3,3-tetra-methylguanidine was added to activate the polyglycerol initiator, thereby promoting ROP. The feed molar ratios of the initiator and ZLL NCA for synthesizing G2-PLL and G3-PLL were designated at 1:120 and 1:240, respectively. The degree of polymerization (DP) for the PLL segment was fixed at 10 for each arm. The deprotection step involves removing the Z groups using a hydrogen bromide solution.

### 1.2 Characterization

The star-shaped polypeptides were characterized by a proton nuclear magnetic resonance (¹H NMR) spectroscopy and a gel permeation chromatography-light scattering (GPC-LS) system. ¹H NMR analysis was conducted on a BRUKER ADVANCE III HD NMR (600 MHZ) using TFA-di and D₂O as the solvents. The GPC-LS analysis was conducted under the operating conditions at 55° C. and 0.8 mL min⁻¹ of flow rate by using a Postnova PN3160 system equipped with a refractive index (RI) detector and a Postnova PN3609 MALS light scattering detector with nine angles. Two Shodex GPC columns (KD-802.5 and KD-804) were used for efficient separation with an eluent (DMF solution with 0.1 M LiBr) and a polystyrene standard (molecular weight: 277,000 g mol-1) for calibration. The G2-PZLL and G3-PZLL polypeptide samples, dissolved in DMF, were passed through a polytetrafluoroethylene Finetech filter (0.45 μm of pore size, 13 mm in diameter) before GPC-LS analysis.

FIGS. 1G to 1H illustrate the chemical structures and synthesis of star-shaped polypeptides. GPC-LS was used to determine the number-average molecular weight (Mₙ) and molecular weight distribution (M_{w}/Mₙ) of G2-PZLL and G3-PZLL, with results summarized in Table 1.

TABLE 1

| | | | | GPC-LS | | |
|---|---|---|---|---|---|---|
| | Feed molar ratio | ¹H NMR | | | M_{w}/ | |
| Polypeptide | Initiator:ZLL NCA | DP | Mₙ | Mₙ | Mₙ | DP |
| G₂-PZLL₁₀ | 1:120 | 10 | 32200 | 32100 | 1.6 | 10 |
| G₃-PZLL₉ | 1:240 | 10 | 64000 | 57000 | 1.5 | 9 |

The DPs were calculated to be 10 and 9 per arm, respectively, consistent with the feed molar ratios. ¹H NMR analysis confirmed these values, with calculated DPs aligning between polyol initiator protons and PZLL phenyl group protons (—C₆H₅).

Additionally, 2D ¹H-¹³C HSQC NMR analysis was conducted on the initiators (PGD G3) and star-shaped PZLL polypeptides (24s-PZLL9). As shown in FIG. 1I, the crossover points, corresponding to the ¹H and ¹³C peaks of primary alcohol (—CH₂OH) and secondary alcohol (—CHOH), disappeared after polymerization, suggesting the successful initiation of polymerization via all the hydroxyl groups promoted by TMG. After deprotection, ¹H NMR showed residual Z groups below 3.0%, indicating successful deprotection. The synthesized star-shaped polypeptides were designated as G2-PLL₁₀ and G3-PLL₉, respectively.

### Embodiment 2: HA/G3-PLL9 Microgels

### 2.1 Preparation of Microgels

To improve targeted delivery, G3-PLL₉ was complexed with HA to form microgels. HA was modified with cysteamine (Cys) to introduce thiol groups via 1-ethyl-3-(3-dimethylaminopropyl) carbodiimide (EDC) and N-hydroxysuccinimide (NHS) activation of carboxyl groups as shown in FIG. 1J. Initially, HA (1.0 mmol) was dissolved in deionized water (DI water) along with EDC (1.2 mmol) and NHS (2.0 mmol) and stirred for 15 minutes to activate the carboxyl groups. Subsequently, cysteamine (0.5 mmol) was added to the reaction mixture, and the reaction was allowed to proceed for 24 h. Upon completion of the reaction, the Cys-modified HA product was dialyzed against DI water for 2 days at 4° C. to remove unreacted cysteamine, EDC, and NHS, followed by freeze-drying to obtain the purified Cys-modified HA. For the preparation of HA/G3-PLL₉ microgels, the Cys-modified HA and G3-PLL₉ solutions with the concentration of 2.0 mg mL⁻¹ were separately prepared in DI water and the G3-PLL₉ solution was slowly added to the Cys-modified HA solution (10 mL) under ultrasonic conditions. The formation of colloidally stable HA/G3-PLL₉ microgels was achieved by controlling the amount of G3-PLL₉ solution added to the Cys-modified HA solution. The highest amount of G3-PLL₉ solution added to the Cys-modified HA solution (10 mL) was determined to be 2.9 mL without causing precipitation. For the resulting HA/G3-PLL, microgel solution, the concentrations of G3-PLL₉ and Cys-modified HA were 0.45 and 1.55 mg mL⁻¹, respectively.

The DoS of Cys grafted on HA was characterized by NMR analysis. The hydrodynamic diameter (particle size), and zeta potential of the HA/G3-PLL₉ microgels were measured using a dynamic light scattering (DLS, Otsuka ELSZ-1000) system. The measurements were conducted in triplicate (n=3) and the autocorrelation functions in the dataset were fitted by employing the cumulant method and CONTIN algorithms.

### 2.2 DoS of Cys-HA & Degradative Responses of Microgel Particles

To enhance the potential clinical applicability of G3-PLL₉, the Cys-modified HA was used to complex with G3-PLL₉ to form microgels via electrostatic interactions as schematic illustrated in FIG. 1F. Notably, the mixing of unmodified HA and G3-PLL, would result in precipitation. The conjugation of Cys onto HA would lower the charge density on HA, which could afford the formation of colloidally stable HA/G3-PLL₉ microgels. Based on the ¹H NMR spectrum, the DoS of Cys was determined to be 39.9%. For the resulting HA/G3-PLL₉ microgel solution, the concentration of G3-PLL₉ is 0.45 mg mL⁻¹. Based on the DLS analysis, the particle size and zeta potential of the microgels were measured to be 355.4±40.4 nm and 39.6±1.4 mV, respectively (FIG. 1K).

The stability of HA/G3-PLL₉ microgels under various enzyme concentrations and pH conditions was investigated. In the presence of hyaluronidase (0.2 mg mL⁻¹), the microgel size decreased to 21% of the original diameter within 15 min and gradually reduced to 7%, reaching a plateau thereafter (FIG. 1L). Under a higher concentration of hyaluronidase (1.0 mg mL⁻¹), the microgel size was rapidly degraded to 7% of their original size (data not shown). These results suggest that HA/G3-PLL₉ microgels can be effectively degraded in the intestinal environment in the presence of hyaluronidase, allowing for the rapid release of G3-PLL₉ to exert its therapeutic effect. The microgel size changes under different pH conditions were evaluated as shown in FIG. 1M to 1O. At pH 7.4, the microgels exhibited minimal changes in size over time, indicating good stability (FIG. 1N). In contrast, at pH 5.1 and pH 8.4, the microgel size remained relatively stable initially and, however, significant degradation of the microgels was observed on day 5 (FIG. 1M and 1O).

### Example 1: Antimicrobial Activity of G3-PLL₉

### 1.1 Time-Kill Kinetics Analysis

The toxigenic C. difficile strain JIK 8284 (tcdA⁺, tcdB⁺, ribotype 027), obtained from Professor Pei-Jane Tsai at National Cheng Kung University (NCKU), was used for antimicrobial evaluation through minimum inhibitory concentration (MIC), minimum bactericidal concentration (MBC), and time-kill kinetics analyses. Briefly, morphologically consistent C. difficile colonies were harvested and standardized to 0.5 McFarland turbidity (1.5×10⁸ colony-forming units [CFUs] mL⁻¹), then diluted 100-fold in brain heart infusion supplement (BHIS) medium. G3-PLL₉ was two-fold serially diluted and mixed with equal volumes of bacterial suspensions in 3-mL test tubes. Controls included vancomycin and BHIS alone. Quality control used C. difficile ATCC® 700057 with vancomycin (0.25-2.0 mg L⁻¹). After anaerobic incubation at 37° C. for 48 h, MIC was determined as the lowest concentration preventing visible growth. Due to the opacity of G3-PLL9, which may hinder the accurate interpretation of MIC readings, MBC was further determined to compare its antimicrobial effects with those of vancomycin. For MBC determination, 100-μL aliquots from test tubes at MIC and higher concentrations were serially diluted, plated on CDC agar, and assessed for a ≥99.9% colony reduction after 48 h. All experiments adhered to standardized microbiological procedures. The MIC and MBC of vancomycin against C. difficile was determined for comparative analysis.

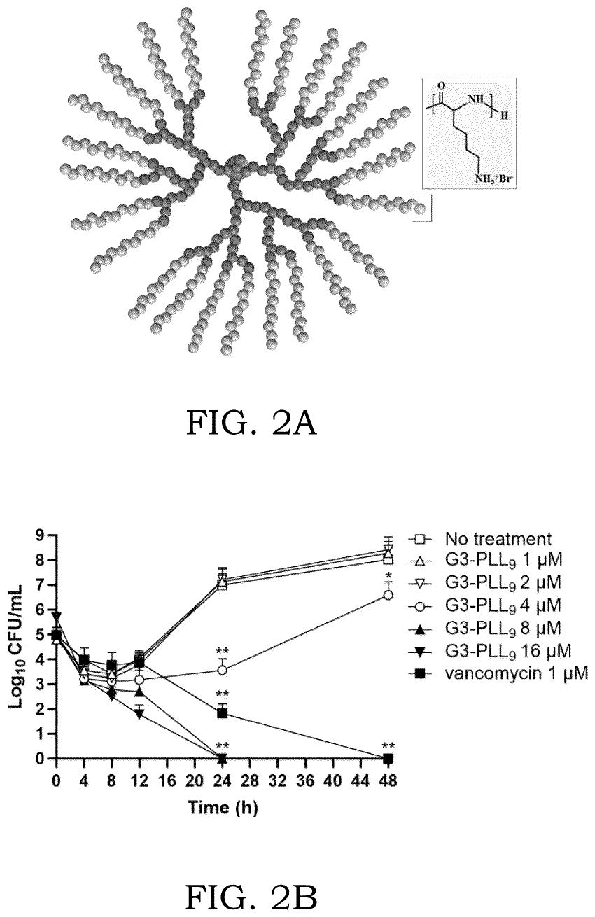

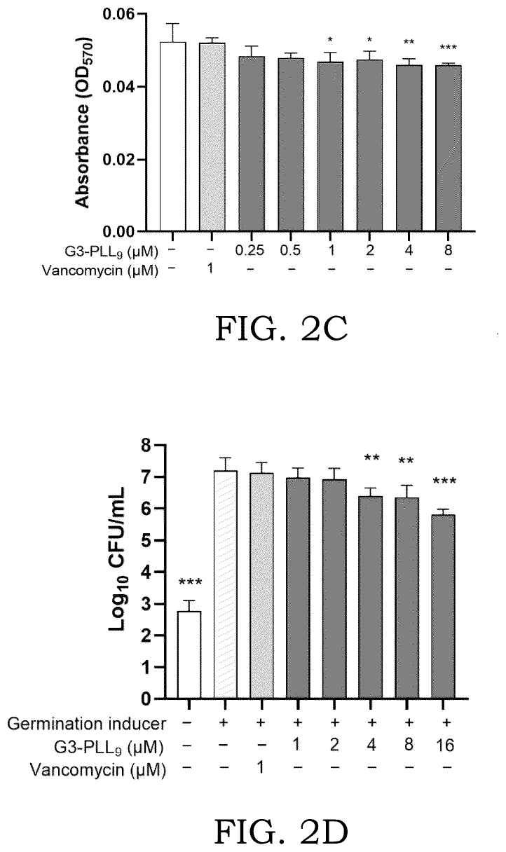

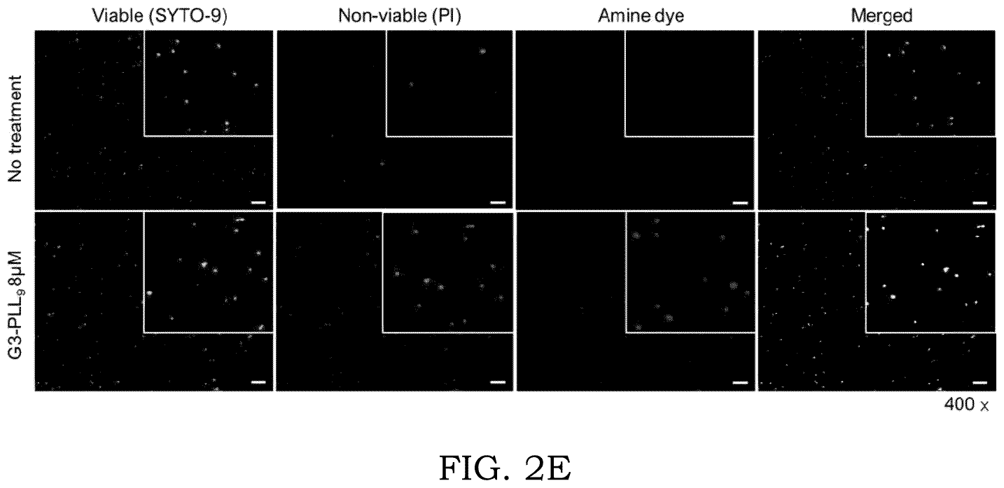

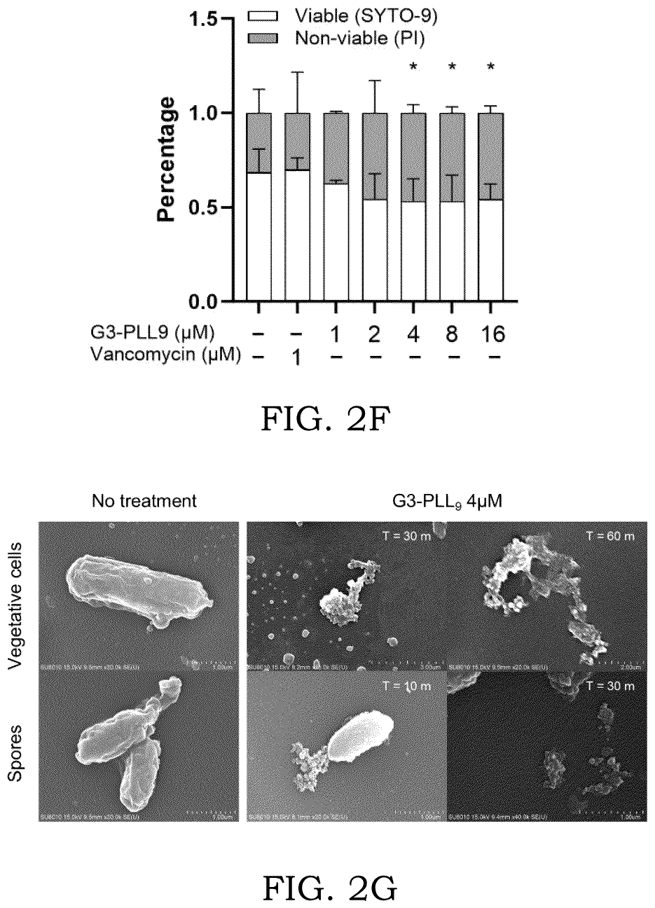

In time-kill analyses, as shown in FIG. 2B, G3-PLL₉ at concentrations of 8 μM or higher consistently demonstrated strong inhibitory effects over 48 h. At 4 μM or lower, partial inhibition was observed within the first 24 h; however, C. difficile recovered beyond this period. Notably, at higher inoculum levels (10⁶-10⁷ CFU mL⁻¹), G3-PLL₉ at 8 μM or higher maintained its inhibitory effects throughout 48 h, contrasting with vancomycin at its MIC (1 μM).

### 1.2 Bacterial Biofilm Quantification

The effect of G3-PLL₉ on C. difficile biofilm (strain JIK 8284) was evaluated by optically measuring the biofilm mass. Biofilms were cultivated in a 96-well plate by inoculating 200 μL of a 0.5 McFarland-adjusted bacterial suspension (diluted with BHIS) into each well and incubating anaerobically at 37° C. for 48 h. Negative controls contained BHIS only. After incubation, wells were washed with phosphate-buffered saline (PBS), air-dried (37° C., 15 min), and treated with G3-PLL₉ (0.25-8 μM) in triplicates. Following another 48 h incubation at 37° C., the supernatants were discarded, and the biofilms were dissolved in ethanol, stained with crystal violet, washed, and the stain intensity was measured as OD₅₇₀ using a microplate reader (SpectraMax® i3x Multi-Mode Detection Platform). Comparative studies included vancomycin and PBS controls.

As shown in FIG. 2C, G3-PLL₉ also disrupted C. difficile biofilm in a dose-dependent manner, a phenomenon not observed with vancomycin at 1 μM. Significantly, G3-PLL₉ dismantled biofilm at sub-MIC concentrations as low as 1 μM. These findings suggest that G3-PLL, exhibits inhibitory effects on C. difficile vegetative cells at concentrations of 4 μM or higher, and on biofilms at concentrations of 1 μM or higher, highlighting its efficacy against both C. difficile vegetative cells and biofilms.

### 1.3 Sporicidal Assay of G3-PLL₉

### 1.3.1 Quantifying Germinated Spores

In short, C. difficile (strain JIK 8284) was cultured anaerobically on CDC agar at 37° C. for 48 h, then adjusted to OD₆₀₀=0.2. A 990-μL bacterial suspension was spread onto a 9-cm dish with 70:30 Sporulation Medium and incubated anaerobically at 37° C. for 7 to 10 days. Spores were harvested using ice-cold sterile water, stored at 4° C. overnight, then washed five times and centrifuged (4000×g, 10 min). The pellet was resuspended in 200 μL ice-cold water and purified via Nycodenz® density gradient centrifugation (10800×g, 1 h). After removing debris, spores were washed, resuspended in sterile water, and stored at 4° C. in the dark.

The sporicidal effect of G3-PLL₉ was assessed by quantifying the number of germinated spores after co-treatment with C. difficile spores. A sublethal heat treatment reduced the population of vegetative cells in the spore stock. The spores were then exposed to G3-PLL₉ for 30 minutes and induced to germinate with taurocholic acid. The mixture was diluted, plated on CDC agar, and incubated anaerobically at 37° C. for 48 h before counting the CFUs. Comparative studies included non-germination and vancomycin controls.

The inhibitory effects of G3-PLL₉ on C. difficile spores were evaluated using multiple complementary methods. At concentrations of 4 μM or higher, G3-PLL₉ induced approximately a one-log reduction in viable bacterial colonies following treatment of spores (FIG. 2D).

### 1.3.2 Fluorescence Microscopy

Fluorescence microscopy assessed C. difficile spore (strain JIK 8284) viability after treatment with G3-PLL₉ and staining using the Live/Dead™ BacLight™ Bacterial Viability Kit (Thermo Fisher Scientific). G3-PLL, was labeled with an amine-reactive dye (Alexa Fluor® 350, Thermo Fisher Scientific) to enhance visualization and specificity before co-treatment with C. difficile spores. Samples were examined with an upright fluorescent microscope (Olympus, BX53). Comparative studies included vancomycin and PBS controls. The proportion of viable and non-viable cells was calculated as the respective cell count divided by the total cell count (viable+non-viable) and expressed as a percentage.

Fluorescence microscopy revealed an increased number of non-viable spores, indicated by red fluorescence, after G3-PLL₉ treatment (FIG. 2E, second column). The polypeptide, conjugated with an amine-reactive blue dye, appeared as white spots in the merged images, suggesting direct interaction with spore surfaces (FIG. 2E, third column). Quantitative analysis of fluorescence images further confirmed a significant increase in non-viable spores at concentrations of 4 μM or higher (FIG. 2F). In contrast, vancomycin (1 μM) showed no observable effect on spore viability (FIGS. 2D and 2F).

### 1.3.3 Scanning Electron Microscopy

Scanning electron microscopy (SEM; JEOL JSM 6700F) was employed to analyze morphological changes in C. difficile cells and spores (strain JIK 8284) following G3-PLL₉ treatment. The cells were treated with G3-PLL, for 10 minutes, 30 minutes, and 1 h to evaluate the changes in their outer surface structures.

SEM images revealed that G3-PLL₉ caused surface roughening of spores within 10 minutes of treatment at 4 μM, followed by disintegration and shrinkage of both spores and vegetative cells (FIG. 2G). These observations suggest that G3-PLL₉ exerts its antimicrobial activity structural disruption of spores and cells.

Data in FIGS. 2B to 2D and 2F are presented as median±interquartile range. *p<0.05, **p<0.01, ***p<0.001. CFU, colony-forming unit, and the statistical significance was evaluated by comparing the non-viable cells of each treatment group to the untreated group (second row) using the Mann-Whitney test.

### Example 2: In Vivo Therapeutic Efficacy

### 2.1 CDI Mouse Model

The therapeutic efficacy of G3-PLL₉ against CDI was evaluated using a previously established mouse model, modified from an earlier protocol. All animal experiments followed ethical guidelines and were approved by the Institutional Animal Care and Use Committee of NCKU (IACUC Approval No.: 112234). Housing conditions included a 13-hour light/11-hour dark cycle (7 AM-8 PM), temperatures of 23±1° C., and humidity levels of 45±15%. After disrupting the gut microbiota with antibiotics, male C57BL/6JNral mice were infected with spores of C. difficile strain JIK 8284 (10⁶ spores per 100 μL). To prevent degradation in the stomach and duodenum, G3-PLL₉ was administered via anal enema. Mice received 30-μL enemas of G3-PLL₉ 0.25, 1.0, 4.0 μM or vancomycin 50 mg/kg/day at 8 h, 24 h, and 48 h post-infection. HA/G3-PLL₉ microgels, designed to facilitate targeted colon delivery, were administered orally at concentrations of 1.0, 2.5, and 5.0 μM in 100-μL volumes. The mock group was not infected with C. difficile and served as a control to monitor changes in the gut microbiome following the antibiotic cocktail treatment. After 52 h, the mice were euthanized for sample collection. In a recurrent CDI model, mice were observed for two weeks post-infection before euthanasia, following the same procedures as in the primary CDI model treated with G3-PLL₉ enemas. Each group consisted of five mice, with experiments conducted in three replicates.

### 2.1.1 G3-PLL₉ Demonstrates Therapeutic Efficacy and a Favorable Safety Profile In Vivo

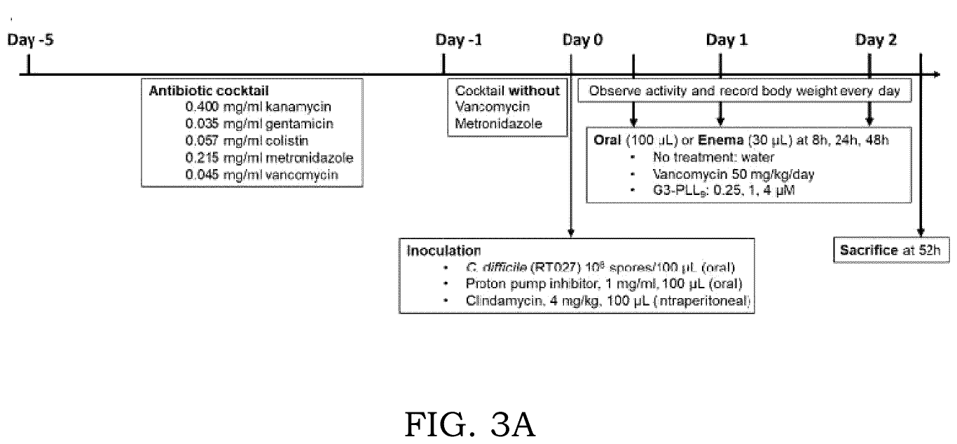

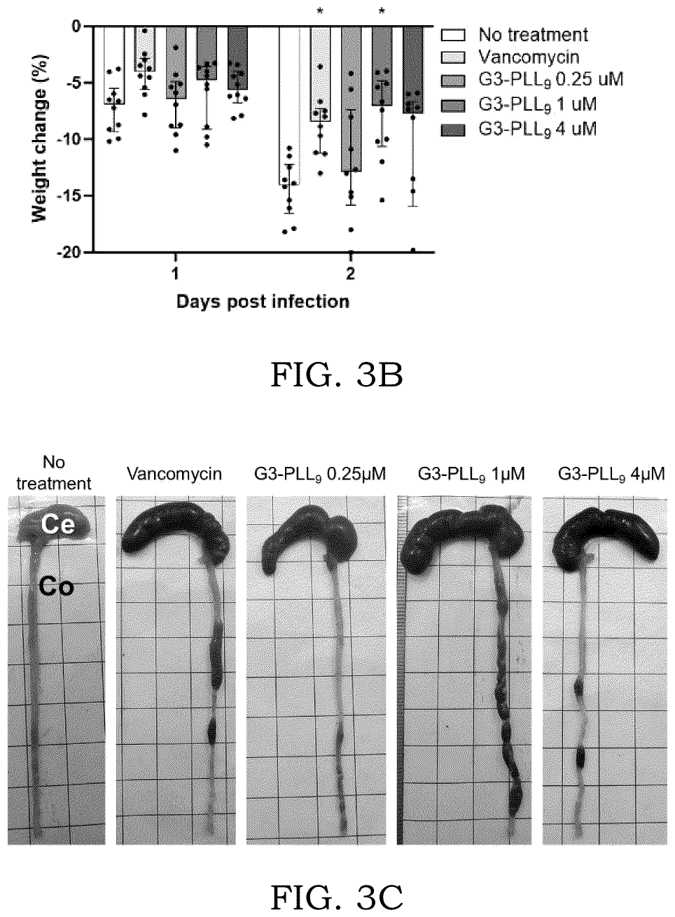

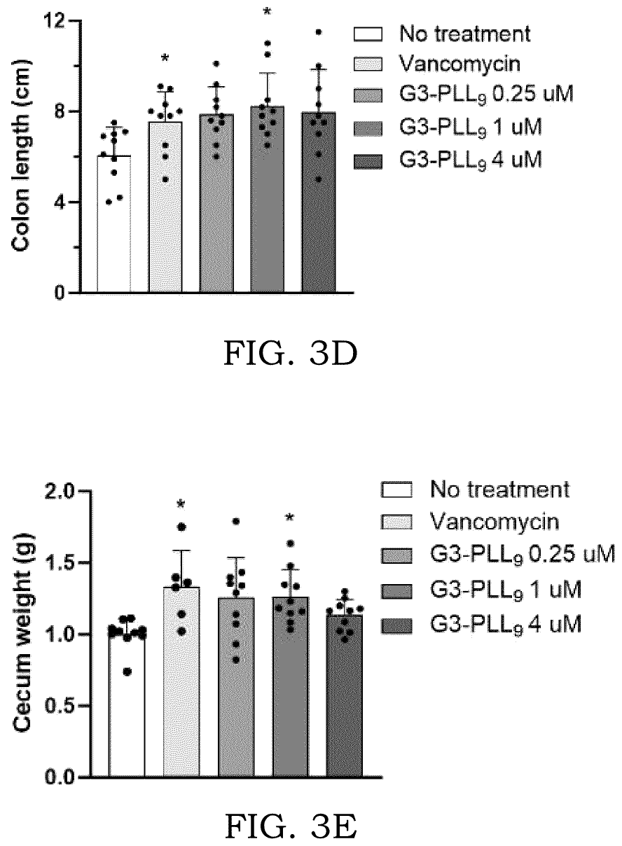

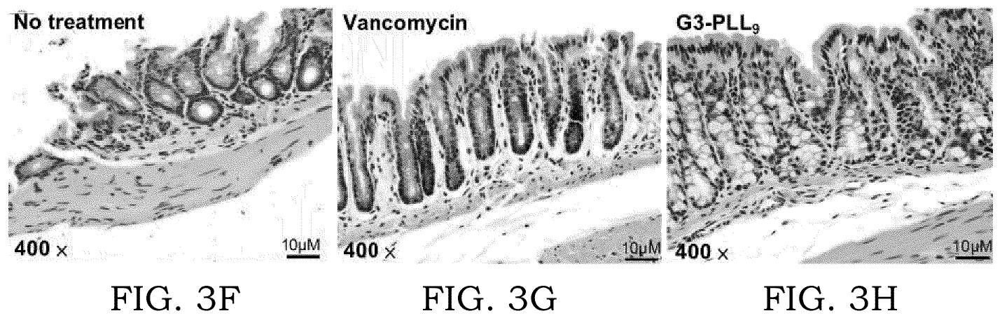

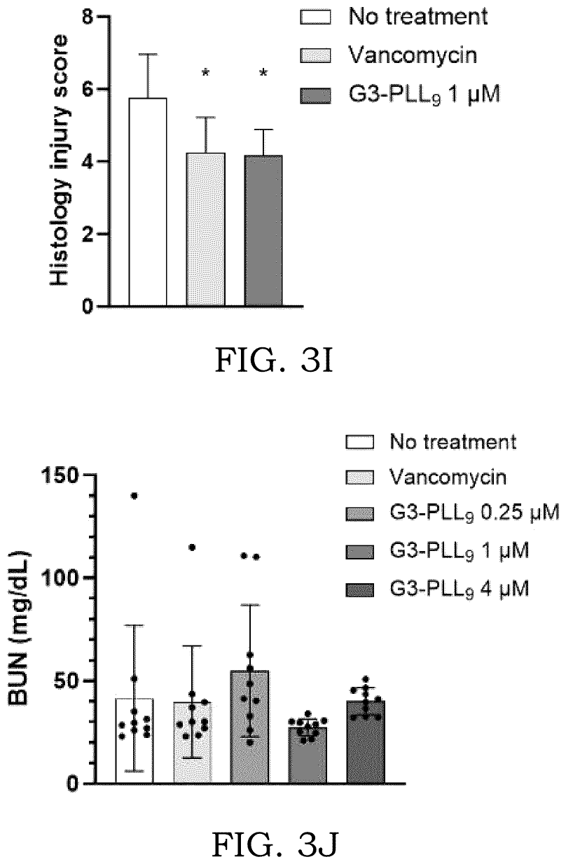

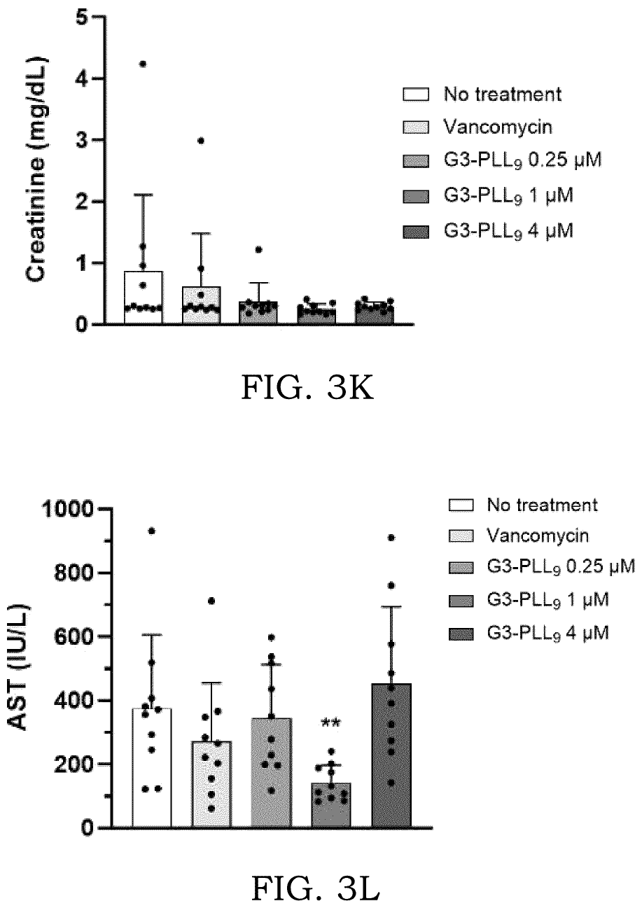

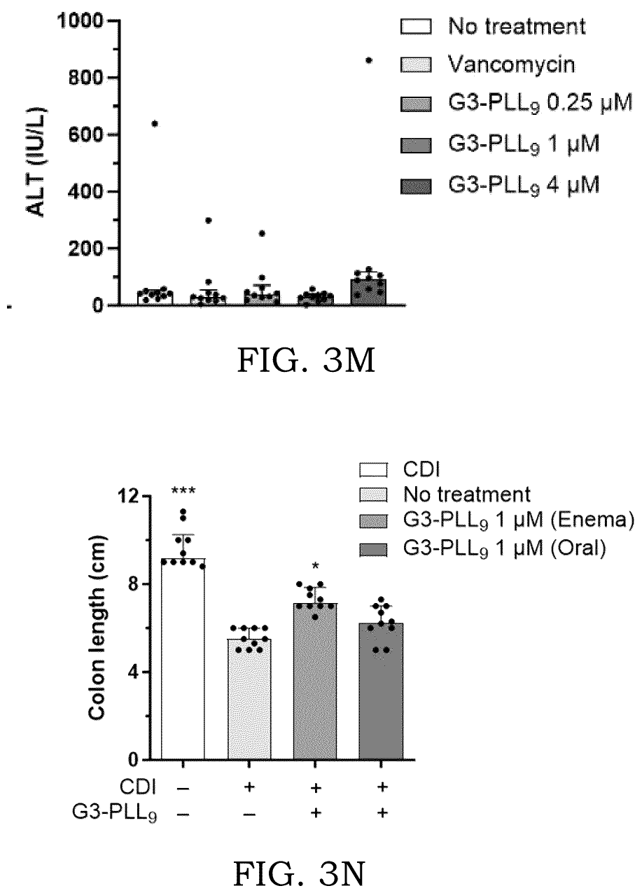

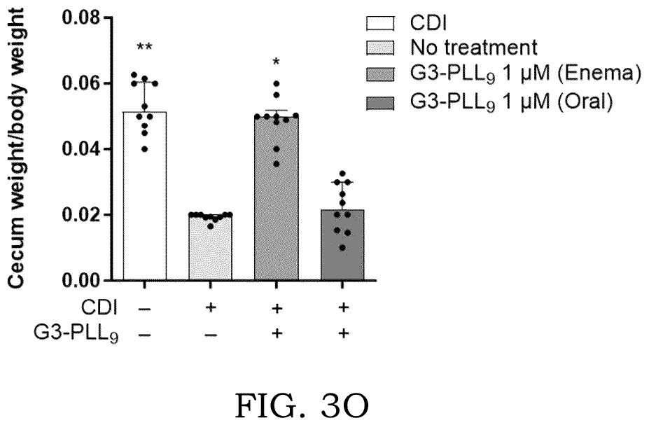

The in vitro antimicrobial activity of G3-PLL₉ against both vegetative cells and spores of C. difficile was established in earlier experiments. Following confirmation that G3-PLL₉ did not induce increased hemolysis at therapeutic concentrations, the in vivo efficacy was evaluated using a mouse model of CDI, and the experimental design could be found in FIG. 3A. G3-PLL₉ was administrated using anal delivery, as, referring to FIGS. 3N and 3O, oral administration of G3-PLL, showed limited effectiveness compared to enema delivery, likely due to degradation by gastric acid.

FIGS. 3B to 3E showed both the G3-PLL₉ (1 μM) and vancomycin groups showed less weight loss and improved symptom severity, colon morphology, colon length, and cecum weight. With reference to FIGS. 3F to I, histological analysis revealed reduced mucosal damage and neutrophil infiltration in both groups. Additionally, FIGS. 3J to 3M demonstrated results of the biochemistry tests, and indicating that G3-PLL₉ (1 μM) resulted in lower aspartate aminotransferase levels compared to other groups, as shown in FIG. 3L. These findings suggest that G3-PLL₉ enema at 1 μM provides therapeutic effects comparable to vancomycin in treating CDI in mice.

Data in FIGS. 3B, D to E, and I to M are shown as median±interquartile range (n=5). Statistical analysis was performed using the Kruskal-Wallis test with Dunn's multiple comparison test relative to the No treatment control. *p<0.05, **p<0.01, ***p<0.005.

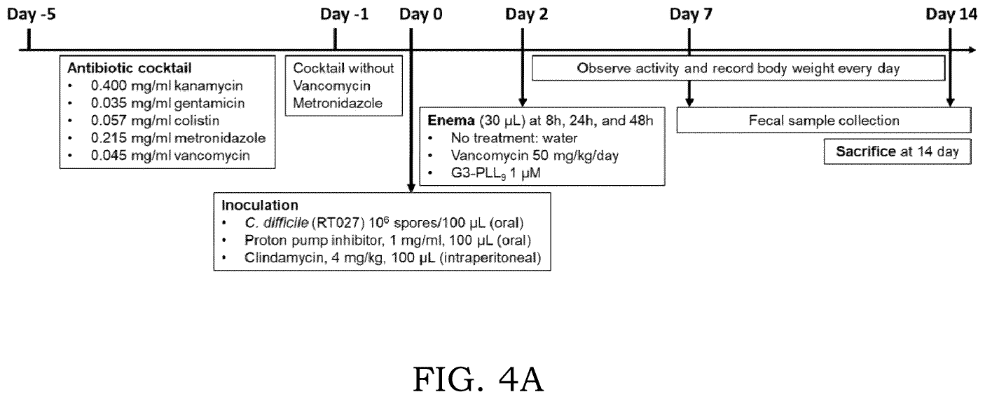

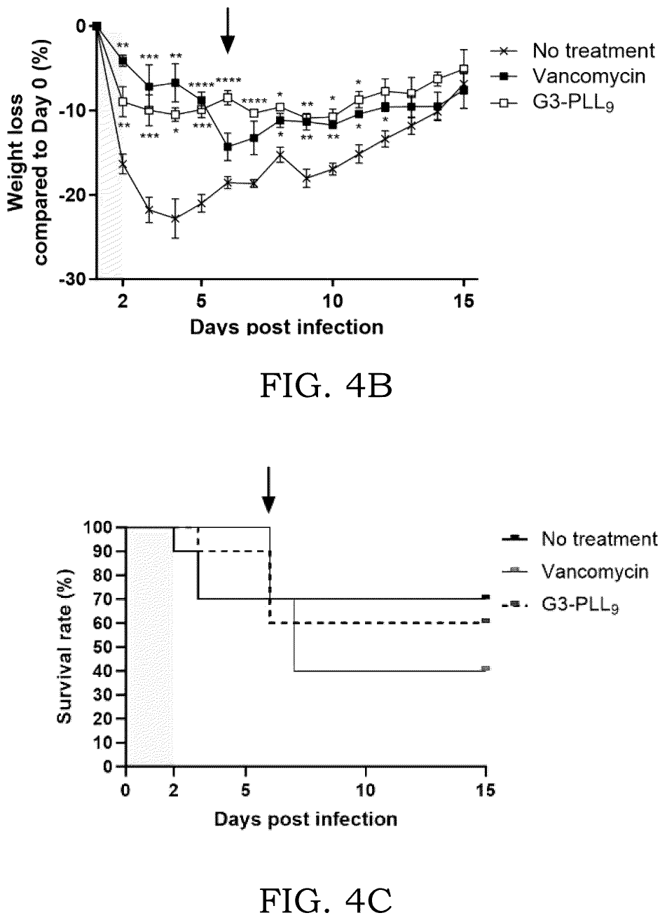

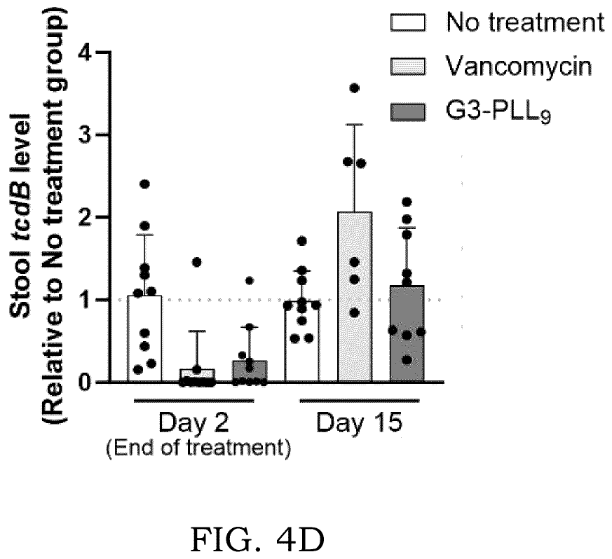

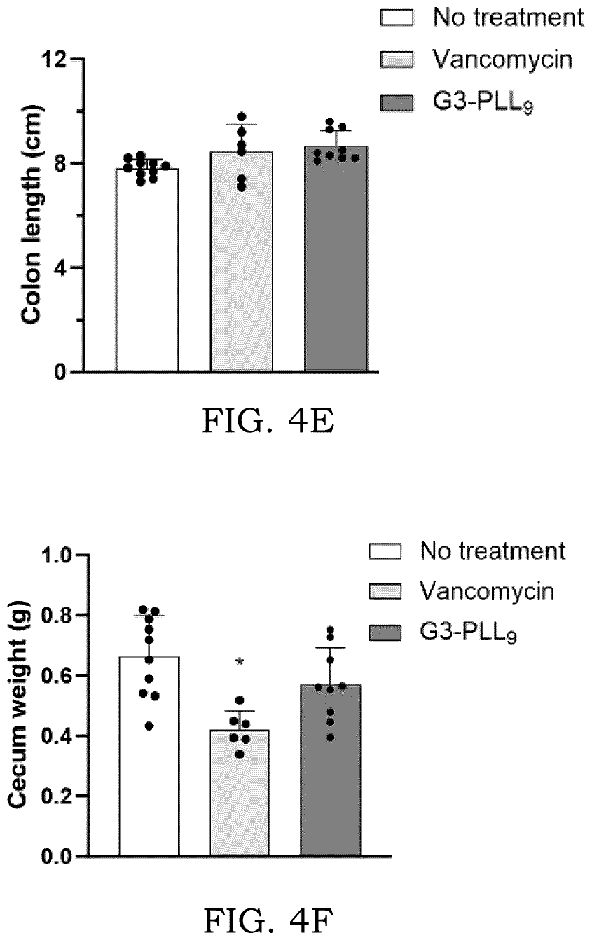

In a recurrent CDI mouse model (FIG. 4A), both the G3-PLL, and vancomycin groups experienced less weight loss during the acute phase within the first two days of infection. However, recurrence occurred approximately one week post-infection, characterized by diarrhea and weight loss (FIG. 4B). Compared to vancomycin, the G3-PLL₉ group exhibited numerically higher overall survival (FIG. 4C), greater body weight retention (FIG. 4B), and increased cecal weight (FIG. 4F). While the vancomycin group had lower toxin levels at the end of treatment, a rebound increase in toxin load was observed by the end of the observation period (FIG. 4D). These findings suggest that G3-PLL₉ may help mitigate CDI recurrence symptoms, potentially by targeting C. difficile spores.

Statistical analysis in FIG. 4B was performed using mixed-effects analysis with Dunnett's multiple comparison test relative to the No treatment control. Data in FIGS. 4C to 4F are shown as median±interquartile range (n=5). For data in FIGS. 4E to 4F, statistical analysis was performed using the Kruskal-Wallis test with Dunn's multiple comparison test relative to the No treatment control. *p<0.05, **p<0.01, ***p<0.005, ****p<0.001.

### 2.1.2 Oral Delivery of Cysteamine-Modified Hyaluronic Acid/G3-PLL₉ Microgel

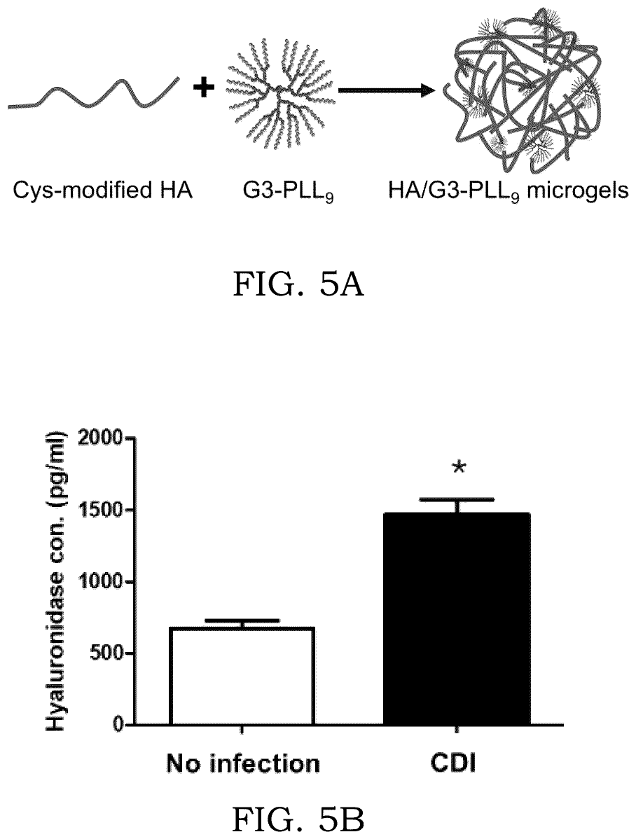

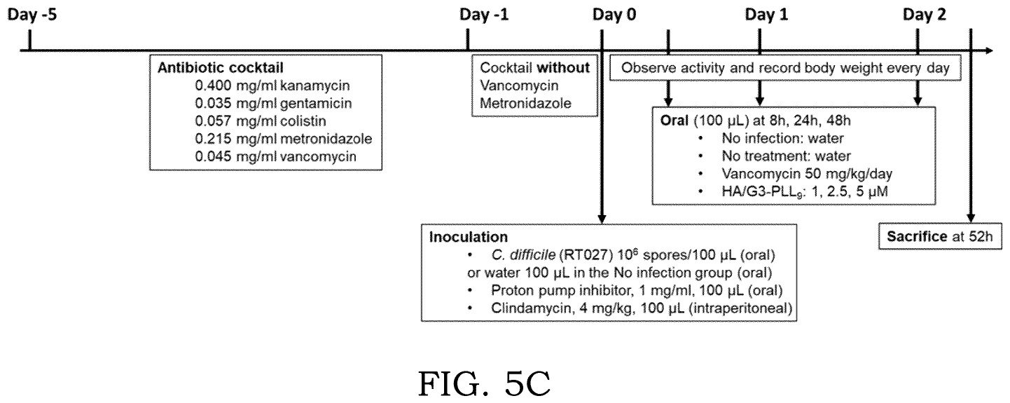

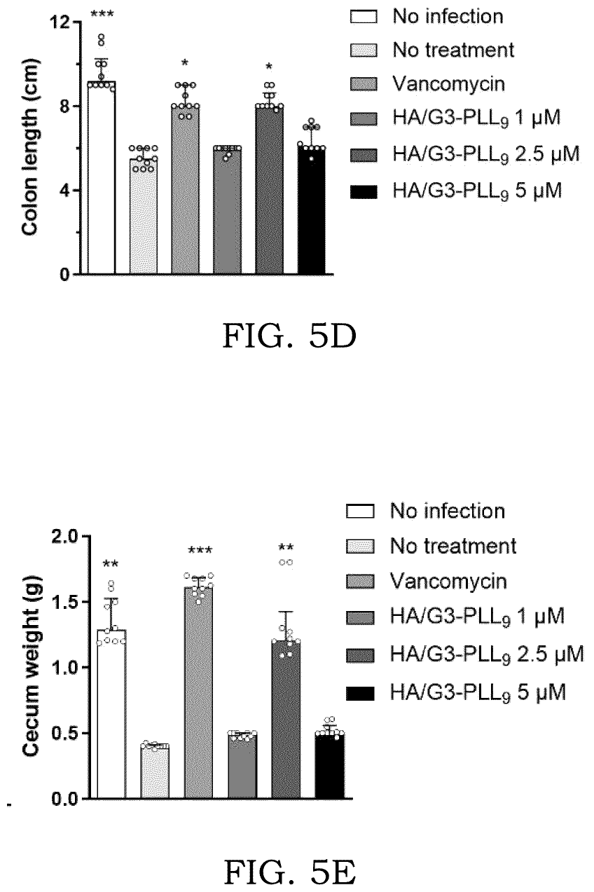

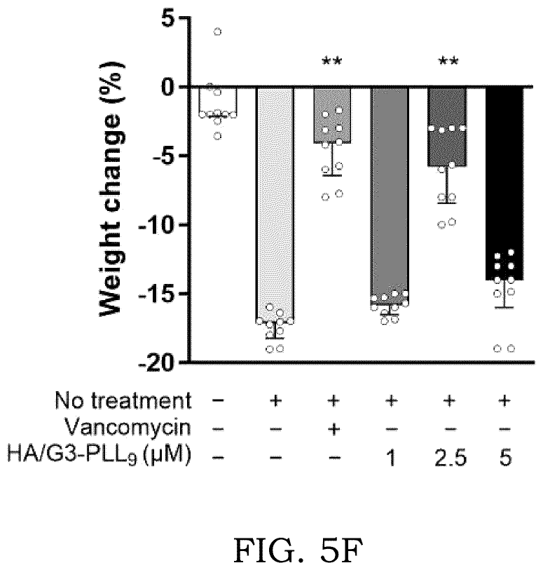

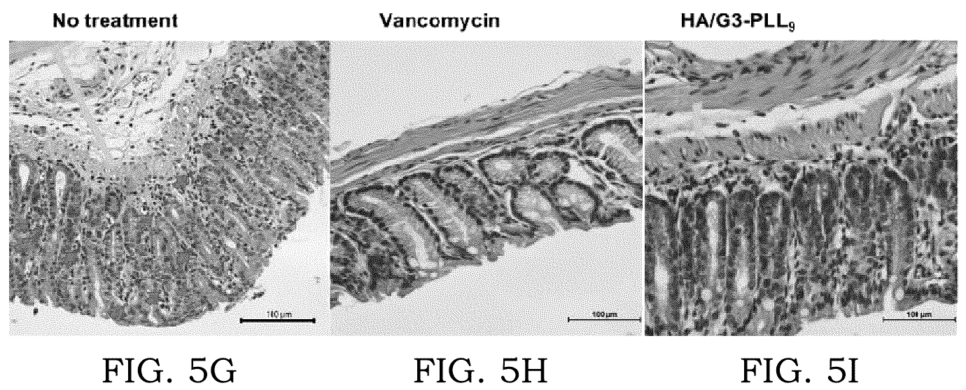

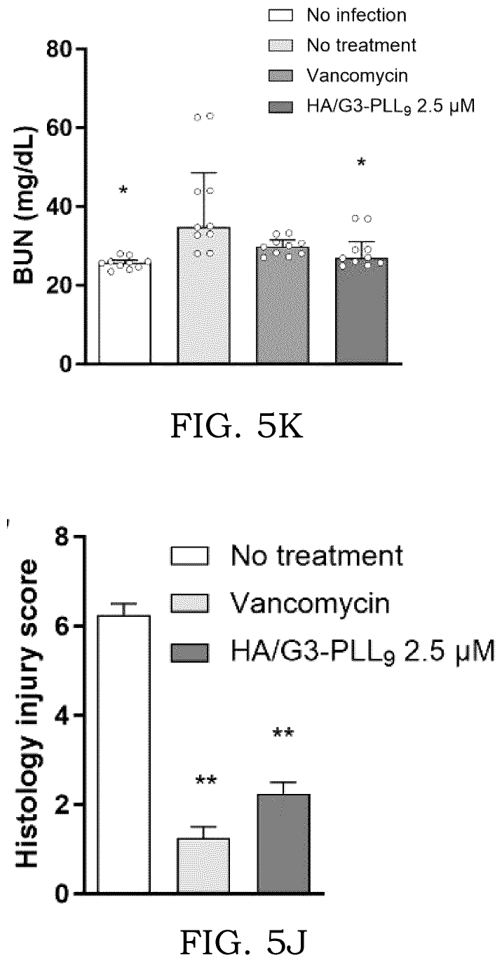

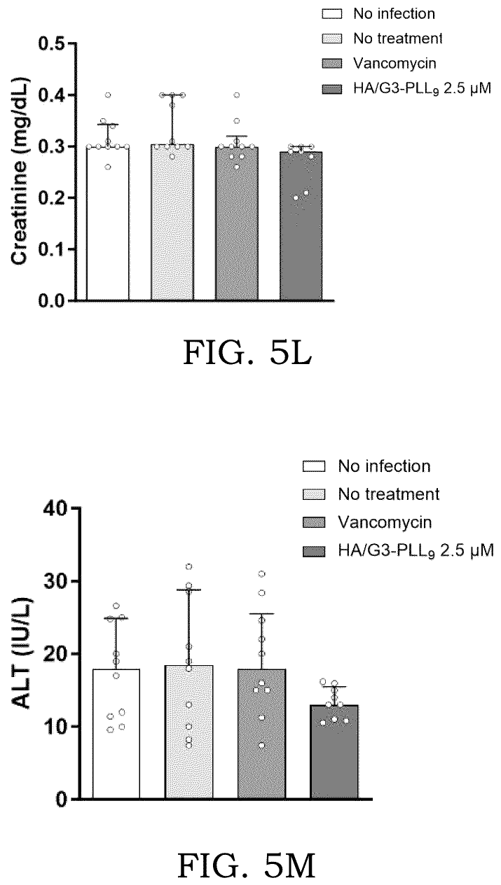

Microgels formed of G3-PLL₉ complexed with the Cys-modified HA is schematically illustrated in FIG. 5A, and subsequently administrated to CDI mouse model. In the CDI mouse model, increased hyaluronidase production was observed in the colon compared to uninfected mice (FIG. 5B). As mentioned in section 2.2 of the Embodiment 2, as the microgels showed sensitive degradative response to hyaluronidase (FIG. 1L), such localized enzyme activity may facilitate the degradation of HA/G3-PLL₉ microgels at the infection site, thereby minimizing off-target effects. HA/G3-PLL₉ microgels were then administered orally following the established protocol for the CDI mouse model (FIG. 5C).

At a concentration of 2.5 μM, as shown in FIGS. 5D to 5J, oral administration of HA/G3-PLL₉ microgels demonstrated therapeutic effects, preserving colon length, cecum weight, and body weight, and reducing tissue damage. Furthermore, with reference to FIGS. 5K to 5M, safety analyses revealed no increased liver or kidney injury compared to other treatment groups (FIG. 5K-M). The results indicate that oral administration of HA/G3-PLL₉ microgels produce similar treatment effects to G3-PLL₉ enema.

Data in FIGS. 5B, 5D to 5F, and 5J to 5M are shown as median±interquartile range (n=5). Statistical analysis was performed using the Kruskal-Wallis test with Dunn's multiple comparison test, comparing treatment groups to the No treatment group. *p<0.05, **p<0.01, ***p<0.005.

2.2 Gut Microbiome Analysis and Fecal C. difficile Toxin Quantification

Total DNA from murine fecal samples was isolated using the High Pure PCR Template Preparation Kit (Roche) following manufacturer's protocol. Microbiome composition was analyzed through next-generation sequencing of the 16S rRNA V3-V4 region using an Illumina MiSeq platform (2×300 nt). Bioinformatic analysis included operational taxonomic unit clustering against the Greengenes database (v13.8), followed by ecological diversity assessment via Shannon index (α-diversity) and Bray-Curtis dissimilarity (β-diversity).

Quantification of toxigenic C. difficile in fecal samples was performed via real-time PCR amplification of the tcdB gene. Universal primers were employed for normalization, and relative quantification was determined using the 2^{-ΔΔct} method.

### 2.2.1 G3-PLL₉ Preserves Gut Microbiota Compared to Vancomycin in CDI Treatment

Preliminary tests indicated that G3-PLL₉ exhibits minimal antimicrobial activity against common gut commensals such as Escherichia coli, Klebsiella pneumoniae, and Staphylococcus aureus, and the results can be found in Table 2.

TABLE 2

| | Minimum bactericidal concentration (μM) | |
|---|---|---|
| Bacteria | G3-PLL₉ | Vancomycin |
| Staphylococcus aureus | 16 | — |
| Escherichia coli | 64 | — |
| Klebsiella pneumoniae | >64 | — |
| Clostridioides difficile | | |
| Vancomycin-resistant | 16 | >64 |
| Metronidazole-resistant | 16 | 8 |
| Ribotype 017 | 8 | ≤1 |
| Ribotype 027 | 8 | ≤1 |

Analysis of fecal microbiota demonstrated differential taxonomic modulation between G3-PLL9 and vancomycin treatments in CDI-affected mice.

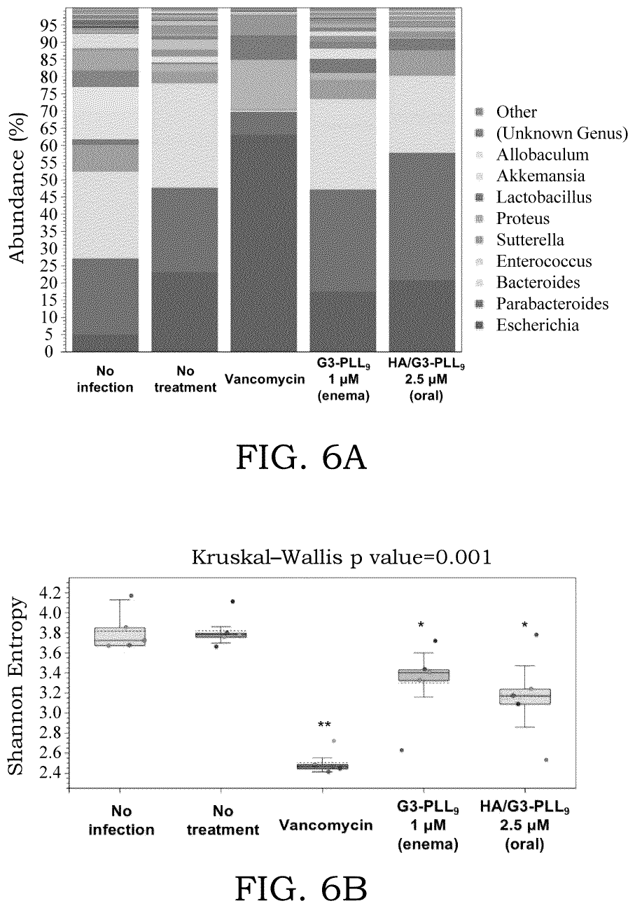

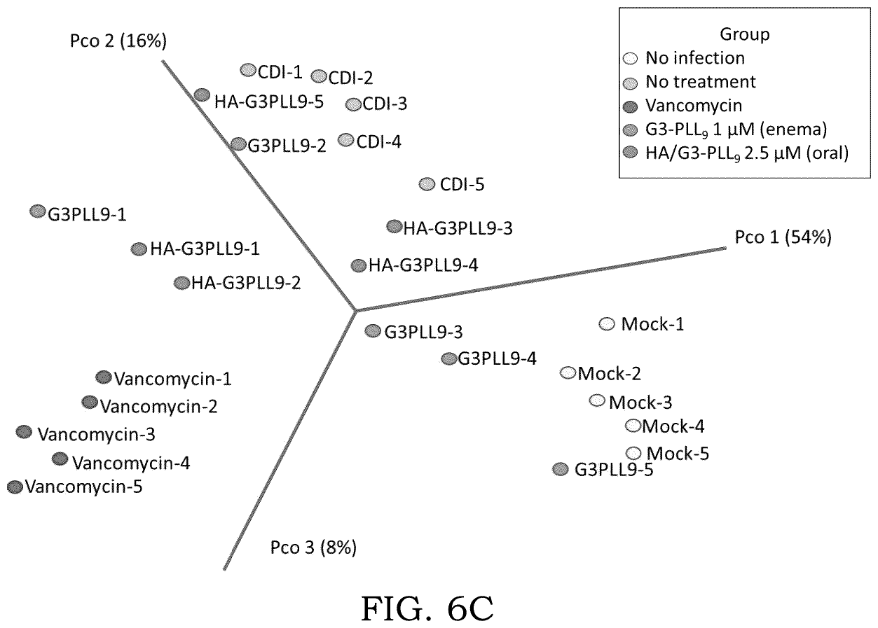

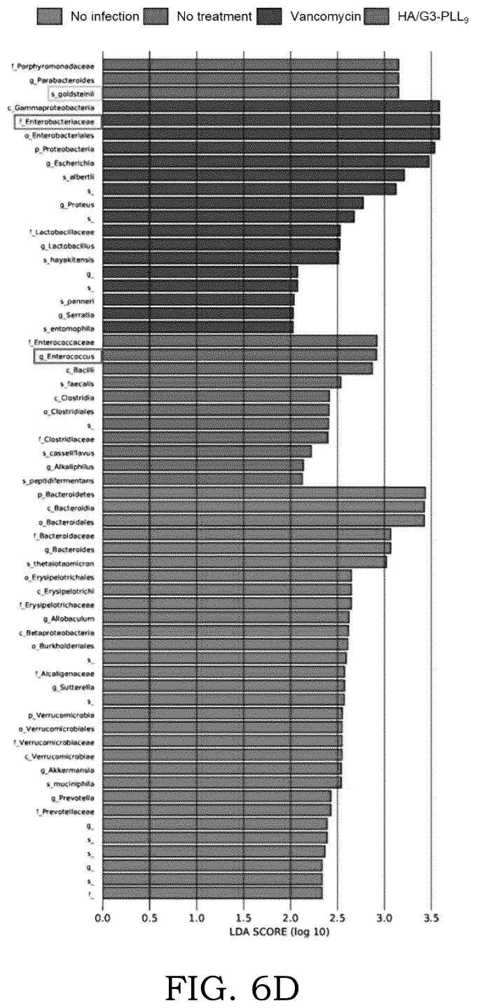

As shown in FIG. 6A, Vancomycin administration led to an enrichment of potentially pathogenic Proteobacteria genera (Escherichia and Proteus), while simultaneously depleting beneficial Bacteroidetes genera (Bacteroides and Parabacteroides). In contrast, FIG. 6B showed that treatments with G3-PLL, and HA/G3-PLL₉ microgels preserved the gut microbiome more effectively, as evidenced by higher alpha diversity metrics, indicating improved species richness and evenness.

Further in view of FIG. 6C, multivariate beta diversity analysis highlighted clear separation among the microbiota compositions of the treatment groups, with G3-PLL₉ and HA/G3-PLL₉ microgels groups showing statistically significant differences compared to the CDI and vancomycin groups. As illustrated in FIG. 6D, linear discriminant analysis further identified key microbial taxa associated with each treatment group, emphasizing the restoration of beneficial bacteria and the reduction of pathogenic species in the G3-PLL₉-treated mice. These results indicated that G3-PLL₉ (enema) and HA/G3-PLL₉ microgels (oral) treatments preserve gut microbiota diversity and composition more effectively than vancomycin, which could have significant implications for promoting long-term gut health and reducing CDI recurrence.

In the present invention, the positively charged antimicrobial block polypeptide (1) demonstrated sporicidal effect on C. difficile in recurrent CDI mouse models. Moreover, when complexed with negatively charged enzyme-targeting macromolecule (2), the gel-form complex prevents degradation of the antimicrobial block polypeptide (1) in the body when administrated orally.

Embodiments of G3-PLL₉ complexed with the negatively charged hyaluronic acid (HA) via electrostatic interactions, enables the formation of microgels. Such complexation enhances not only biocompatibility but also therapeutic efficacy via targeted and on-demand delivery. As a glycosaminoglycan with immunomodulatory properties, HA facilitates targeted delivery and minimizing off-target effects. HA-based delivery systems are particularly advantageous for gastrointestinal therapy, as HA-integrated microgels withstand stomach acidity and release the therapeutic payload in the inflamed colon through hyaluronidase-mediated degradation. This feature ensures precise delivery of bioactive agents to diseased sites, making HA an ideal component for oral delivery systems.

Accordingly, the complexation of the antimicrobial block polypeptide and the enzyme-targeting macromolecule, as exemplified of HA/G3-PLL₉ microgels, offers a promising bacterial gastrointestinal infection-targeted therapy. Examples in the present invention indicates that, in a C. difficile-infected inflamed-colon, the therapeutic composition is capable of eradicating C. difficile along with its spores and biofilms, diminishing persistent colonization and CDI recurrence.

What is claimed is:

1. A antimicrobial block polypeptide, comprising:
a first positively charged peptide segment consisting of 5 to 20 substituted or unsubstituted amino acids selected from the group consisting of L-lysine, L-arginine, L-histidine L-omithine, and L-homoarginine.

2. The antimicrobial block polypeptide of claim 1, being a linear block polypeptide.

3. The antimicrobial block polypeptide of claim 1, being a branched block polypeptide or a star-shaped block polypeptide, further comprising a second positively charged peptide segment consisting of 5 to 20 substituted or unsubstituted amino acids selected from the group consisting of L-lysine, L-arginine, L-histidine L-omithine, and L-homoarginine, wherein:
the star-shaped block polypeptide comprising;
a polyol initiator or a dendrimer initiator as a core; and
at least 3 arms radiating from the core, wherein the first positively charged peptide segment links to at least one arm of the 3 arms, and the second positively charged peptide segment links to at least one another arm of the 3 arms.

4. The antimicrobial block polypeptide of claim 3, wherein:
the polyol initiator comprises 1,1,1-tris (hydroxymethyl)propane or dipentaerythritol;
the dendrimer initiator comprises a poly (amidoamine) (PAMAM) dendrimer, a polylysine dendrimer, a poly (propylene imine (PPI) dendrimer, a polyester dendrimer, a polyglutamic acid dendrimer, a polyaspartic acid dendrimer, a polyglycerol dendrimer, and a polymelamine dendrimer.

5. The antimicrobial block polypeptide of claim 3, wherein:
the first positively charged peptide segment is consisting of substituted or unsubstituted L-lysine or L-homoarginine;
the second positively charged peptide segment is consisting of substituted or unsubstituted L-lysine or L-homoarginine.

6. The antimicrobial composition of claim 3, having 3 to 24 arms.

7. A therapeutic composition, comprising the antimicrobial block polypeptide of claim 1.

8. The therapeutic composition of claim 7, being an enterally absorbable composition.

9. The therapeutic composition of claim 7, further comprising an enzyme-targeting macromolecule comprising:
a backbone; and
a negatively charged functional group residing on the backbone, wherein the antimicrobial block polypeptide docks on the enzyme-targeting macromolecule via electrostatic interaction between the first positively charged peptide segment and the negatively charged functional group.

10. The therapeutic composition of claim 9, wherein the backbone comprises glycosaminoglycan selected from a group consisting of Hyaluronic Acid (HA), Chondroitin Sulfate (CS), Dermatan Sulfate (DS), Keratan sulfate (KS), Heparan sulfate (HS), and Heparin (HP).

11. The therapeutic composition of claim 9, wherein the negatively charged functional group is selected from a group consisting of thiol, thiolate, thioacid, thiophosphate ester, and selenol.

12. The therapeutic composition of claim 9, being an oral composition.

13. A method for treating bacterial gastrointestinal infection, comprising:
administering an effective amount of the therapeutic composition of claim 7 to a subject in need thereof, wherein the bacterial gastrointestinal infection comprises pseudomembrane colitis, gastroenteritis, diarrhea, toxic megacolon or gastrointestinal perforation

14. The method of claim 13, wherein the effective amount is from 0.5×10⁻¹⁰ mol/kg to 5.0×10⁻¹⁰ mol/kg bodyweight of the subject, or from 2.0×10⁻¹⁰ mol/kg to 16.5×10⁻¹⁰ mol/kg bodyweight of the subject.

15. The method of claim 13, wherein the therapeutic composition reduces population, spore number or biofilm of a pathogen inducing the bacterial gastrointestinal infection.

16. The method of claim 15, wherein the pathogen is selected from group consisting of Clostridium botulinum, Clostridium difficile, Clostridium perfringens, Clostridium aldenense, Clostridium asparagiforme, Clostridium bolteae, Clostridium citroniae, Clostridium clostridioforme, Clostridium lavalense, Clostridium nexile, Clostridium populeti, and Clostridium symbiosum.

17. The method of claim 13, wherein the therapeutic composition is administered by enema to the subject.

18. The method of claim 13, wherein the therapeutic composition further comprises an enzyme-targeting macromolecule comprising:
a backbone; and
a negatively charged functional group residing on the backbone, wherein the antimicrobial block polypeptide docks on the enzyme-targeting macromolecule via electrostatic interaction between the first positively charged peptide segment and the negatively charged functional group.

19. The method of claim 18, wherein:
the backbone comprises glycosaminoglycan selected from a group consisting of Hyaluronic Acid (HA), Chondroitin Sulfate (CS), Dermatan Sulfate (DS), Keratan sulfate (KS), Heparan sulfate (HS), and Heparin (HP);
the negatively charged functional group is selected from a group consisting of thiol, thiolate, thioacid, thiophosphate ester, and selenol.

20. The method of claim 18, wherein the therapeutic composition is administered orally to the subject.
